(19) United States
(12) Patent Application Publication
(10) Pub. No.: US 2002/0037868 A1
(43) Pub. Date: Mar. 28, 2002

(54) Method for detecting hepatitis C

(75) Inventors: Agata Budkowska, Clamart (FR); Patrick Maillard, Montlhery (FR); Christian Bronnert, Fresnes (FR); Pierre Gounon, Noiseau (FR); Jadwiga Nitkiewicz, Komorow (PL); Radu Grainic, Jouy En Josas (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: INSTITUT PASTEUR, Paris Cedex (FR)

(21) Appl. No.: 09/880,945

(22) Filed: Jun. 15, 2001

Related U.S. Application Data
(63) Continuation-in-part of application No. 09/549,685, filed on Apr. 14, 2000.
(60) Provisional application No. 60/129,319, filed on Apr. 14, 1999.

(51) Int. Cl. C12Q 1/70; G01N 33/53; G01N 33/537; G01N 33/543; A61K 31/70; A01N 43/04; C12P 19/34

(52) U.S. Cl. 514/44; 435/5; 435/7.95; 435/91.33; 435/975

(57) ABSTRACT

The present invention provides methods of detecting hepatitis C virus.

### BACKGROUND OF THE INVENTION

1. Field of the Invention

The present invention relates to a methods for detecting hepatitis C virus.

2. Description of the Background

Hepatitis C virus (HCV) is an enveloped positive strand RNA virus, recognized as the major etiologic agent of blood-borne and sporadic non-A, non-B hepatitis. Due to the propensity of this virus to cause chronic infections, and its association with liver cirrhosis and hepatocellular carcinoma, HCV is a significant world-wide health problem.

HCV has a single strand positive sense RNA genome of approximately 9,400 nucleotides in length. The virus has a lipid-containing envelope that is chloroform-sensitive and appears to be necessary for replication. HCV is similar to members of the Flaviviridae family in overall genome organization and in the presumed mechanism of replication. Particularly, HCV genome codes for a single polyprotein precursor of about 3,000 aminoacids that is cleaved into a series of proteins including capsid, two envelope proteins E1 and E2 and 7 putative non-structural proteins some of which are involved the polyprotein processing. Although the entire HCV genome has been sequenced, see Chiron patents: EP 318216, EP 388,232 and PCT WO 90/1443, and the viral proteins and their processing have been well characterized in vitro, little is currently known about the mechanism of HCV infection which leads to viral persistence despite a broad immunological response to viral structural and non-structural proteins. Current diagnoses of HCV infection are based on the detection of viral RNA in serum by polymerase chain reaction (PCR) and antibodies against HCV components by the assays involving multiple HCV recombinant proteins and/or synthetic peptides. However, there are no available diagnostic assays for detection of the structural proteins of circulating virus. The polypeptide composition, antigenic structure of the virion and number of the possible viral serotypes remain unknown.

Putative HCV virion is about 50-60 nm in diameter and is composed of a viral envelope and a 33 nm core. HCV core protein is a highly basic and is mapped to the first 191 aa residues of the HCV polyprotein. This region is well conserved between different HCV isolates and genotypes and shows high degree of homology with nucleocapsid proteins of other flaviviruses. Viral encapsulation requires the self-association and the capacity to interact with the viral RNA. The interaction sites with homologous and heterologous RNA has been mapped to the N-terminal region of the core protein, whereas the main homotypic interaction domain has been mapped to the tryptophan rich aa sequence (73-108). The hydrophobic signal sequence for translocation of El protein into the endoplasmic reticulum is located in the C-terminal part aa (170-191) and is apparently cleaved by proteases associated with cellular membranes at aa 172. Besides its role in viral replication. HCV core protein has many important biological functions, such as modulation of transcription from several cellular promoters, suppression of the HBV gene expression, interaction with the cytoplasmic tail of lymphotoxin receptor and others.

With equilibrium centrifugation and immunoprecipitation studies, it has been demonstrated that HCV populations in serum consist of low density virions associated with P-lipoproteins which are infectious in cultured cells and of the high density fraction that might contain either immune complexes or naked HCV nucleocapsids. Although, several groups have reported the detection of the core antigen by immunological methods in virus-enriched serum samples from HCV-infected individuals after detergent treatment, no free core antigen has yet been isolated from serum and characterized immunochemically.

To date, the two basic tests for HCV are i) PCR, and ii) detection of antibodies in patient serum. However, a need exists for an improved means of detecting HCV.

The present invention provides an evidence that viral particles with the physiochemical, morphological and antigenic properties of non-enveloped HCV nucleocapsids are present in the plasma of HCV-infected individuals. These particles have a buoyant density of 1.32-34 g/ml in CsCl, are heterogeneous in size (with predominance of particles of 38 43 or 54-62 nm in diameter on electron microscopy) and express on their surface epitopes located in the amino-acid sequence (24-68) of the core protein. Similar, nuclecocapsid-like particles are also produced in insect cells infected with recombinant baculovirus bearing c-DNA for structural HCV proteins.

Circulating core particles are reactive with MAbs despite the presence of human anti-HCV antibody in the plasma and serum samples. Human anti-core antibodies from the sera of HCV-infected individuals do not compete with mouse MAbs for these antigenic sites, probably due to the large differences in affinity demonstrated by Surface Plasmon Resonance Analysis.

The principles of assays used in this invention for detection of HCV core are different from all other assays published before (1,19,32,33,38.39.40) or recently commercialised (Ortho Diagnostics) which use detergents or denaturing agents for pre-treatment of serum samples or in a “sample diluent”. These assays quantify mainly the core protein from denatured HCV virons. we could detect HCV core antigen was directly by immunological assays (ex. ELISA in several, fresh, unfractionated and untreated serum and plasma samples. Preliminary results have shown that 37/40 serum samples positive for HCV markers by routine serological methods tested positive in direct ELISA for HCV core antigen(ELISA readings >0.4) whereas 15/18 sera without serologcai markers of HCV infection were negative for the presence of circulating cores (ELISA readings <0.4).

To confirm these findings we were also able to isolate HCV core particles directly from plasma of HCV carriers by affinity chromatography with anti-core antibodes bound to the solid support. Thus, HCV core epitopes are naturally present on circulating HCV particles and can be detected directly with immunological assays involving MAbs, not only in the initial infection phase (window period) but also, as shown here, during chronic phase of disease, even if anti-HCV antibodies are present in the serum. At least three epitopes were found to map to the sequence between aa (24-68) of the serum core particle and this sequence seems to be well conserved in different HCV genotypes (FIG. 9). The detection of circulating, envelope-free HCV nucleocapsids in serum may have potential diagnostic applications.

### SUMMARY OF THE INVENTION

Accordingly, it is an object of the present invention to provide a method of directly detecting HCV in serum of a patient, which represents a surprising improvement over the conventional tests for HCV.

It is also an object of the present invention to provide a method of detecting non-enveloped nucleocapid or non-enveloped core protein of HCV in serum of a patient.

The above objects and others are provided by a method of directly detecting hepatitis C virus in serum of a patient by detecting the virus with primers corresponding to viral RNA encoding core protein which RNA is a light fraction of the total viral RNA, the light fraction being isolated after ultracentifugation in a CsCl gradient of human serum containing HCV virus, the light fraction containing most of the circulating infectious HCV virus particles, which method entails precipitating RNA, effecting reverse transcription and then effecting amplification with the primers described herein.

It is also an object of the present invention is a method of detecting the presence of HCV particles in patient's serum or plasma without chemical pretreatment comprising:

contacting a patient's serum to be tested with a solid phase coated with a first antibody which is directed against HCV core protein;

adding at least one second labeled antibody directed against the HCV core protein, wherein said second antibody can be the same or different from said first antibody; and

detecting the presence or absence of a immune complex formed between said first antibody, said HCV core protein, and said second antibody wherein the presence of said immune complex indicates the presences of HCV particles in the patient's serum or plasma.

It is also an object of the present invention to provide a method of preparing a nucleocapsid-like particle recognized by at least one antibody selected from the group consisting of an antibody recognizing the amino acid region 24-37 of the core protein; an antibody recognizing the amino acid region 40-53 of the core protein; and an antibody recognizing the amino acid region 45-68 of the core protein, wherein said process comprises:

introducing a HCV-1 core gene into a eukaryotic host cell;

culturing the transfected eukaryotic cell for under conditions suitable for the expression of said HCV-1 core gene; and

separating a nucleocapsid-like particle from said host cell.

It is also an object of the present invention to provide a detection kit for HCV infection comprising:at least antibody which binds to the nucleocapsid-like particles; and reagents for labeling and visualization of a reaction between the serum or plasma of a patient to be tested for HCV infection and said at least one antibody.

### BRIEF DESCRIPTION OF THE DRAWINGS

FIG. 1 illustrates HCV-RNA determination by RT-PCR and b-DNA assay.

FIG. 2A illustrates the activity of monoclonal antibody (MAb)VT against HCV core protein.

FIG. 2B illustrates the activity of monoclonal antibody (MAb) 39-72 against HCV core protein.

FIG. 3A illustrates a mapping of epitopes recognized by MAb 39-72 using a panel of synthetic peptides.

FIG. 3B illustrates an epitope analysis of HCV core protein.

FIG. 4 illustrates that MAb VT and MAb 39-72 recognize different, non-overlapping epitopes of HCV core protein.

FIG. 5 illustrates the results of ELISA for HCV-core protein.

FIG. 6 illustrates lack of inhibition of MAb 39-72 used in the assay for core antigen by human globulins containing anti-core antibodies.

FIG. 7 illustrates the results of Western-Blot analysis of the anti-HCV-core IgM MAb.

FIG. 8.

Epitope specificity of MAb ACAP27 (A) MAb VT (B) and human anti-HCV globulins (HCIG, C) determined with a panel of synthetic core peptides.

FIG. 9.

(A). Isolation of naturally occurring HCV core particles from concentrated plasma by isopyenic centrifugation in a CsCl gradient..

(B). Isolation of nucleocapsids from putative HCV virions by detergent treatment. A sample (1.5 ml) of the fraction of the gradient banding at a density of 1.10 g/ml (shown in A) and corresponding to the HCV-RNA peak was treated with 0.5% Tween-80 and centrifuged in a CsCl gradient as shown in (A).

Fractions (0.7 ml) were tested for HCV RNA by RT-PCR, and for HVC core antigen by ELISA.

FIG. 10.

Analysis of HCV nucleocapsids by electron microscopy.(A) direct staining of virus particles with 1% uranyl acetate, (B-H) virus particles were adsorbed on anti-core MAb-coated microscope grids and were stained with 1% uranyl acetate.

(A) Virus particles isolated from serum by affinity chromatography on anti-core MAb ACAP27 bound to the Afli-gel Hz. Insert: larger particle of 54 nm in diameter.

(B and C) Virus particles of 54-62 in diameter observed in core antigen-positive fractions from the CsCl gradient, in addition to the 38-43 nm particles identical to those shown in A.

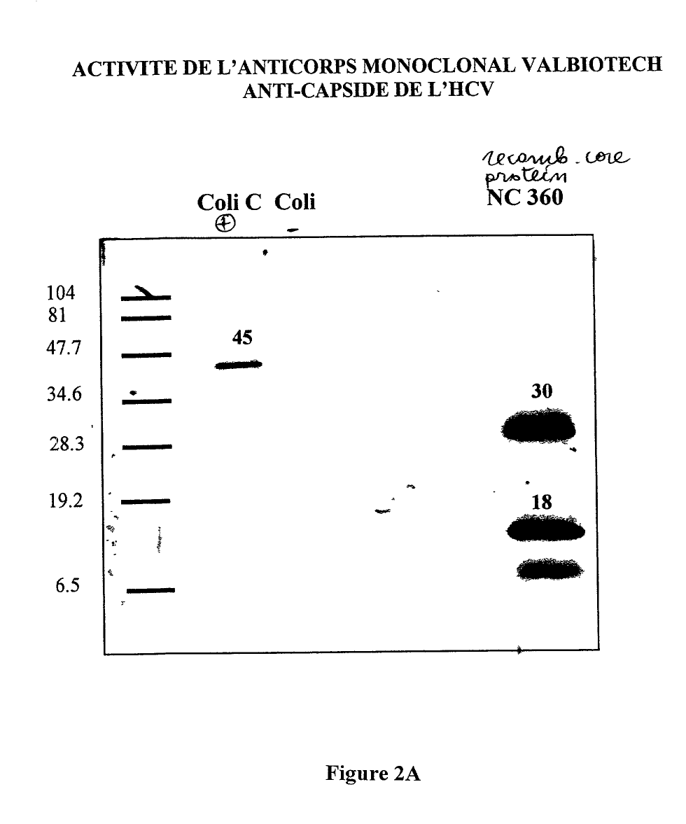

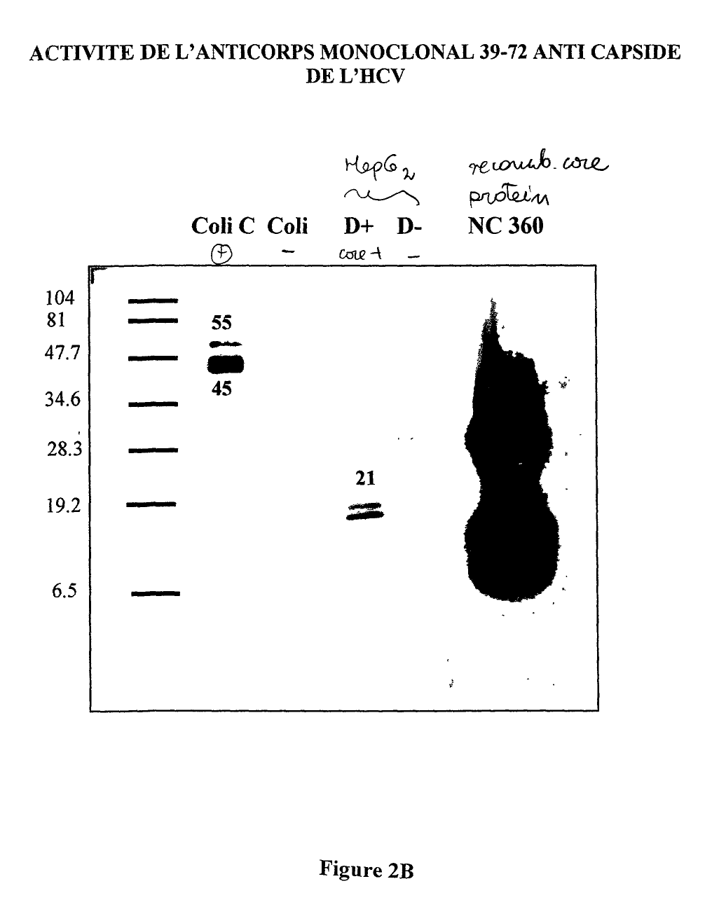

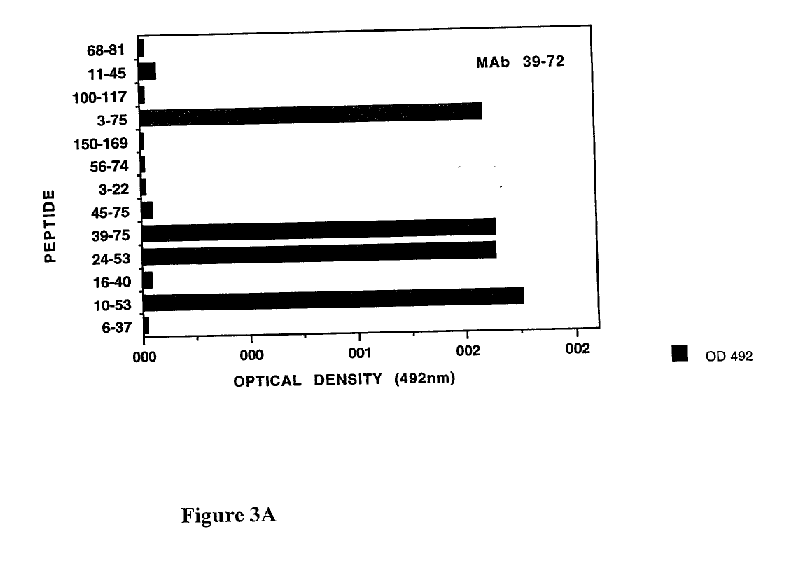

(D, E and F) HCV nucleocapsids isolated from the light fraction of the gradient (density 1.10 g/ml) by treatment with Tween-80 (as shown in FIG. 2B). Most particles are 38-43, in diameter but larger particles (shown in F and with an arrow in D) were also observed.

(G and H) Virus-like particles isolated from baculovirus-infected insect cells. (G) Free nucleocapsid-like particles banding in a CsCl gradient at a density of 1.35-1.36 g/ml in CsCl and

(H) membrane-bound nucleocapsid-like particles banding at a density of 1.25 g/ml in CsCl. Bars in all figures indicate 100 nm.

FIG. 11.

Reactivity of MAb 1/1, generated by immunisation with viral particles purified from plasma:

(A) with the recombinant core protein NC 360 and (B) with a panel of synthetic core peptides. in ELISA.

FIG. 12.

Affinity-capture RT-PCR performed on fresh unfractionated serum samples from HCV carriers. M- molecular mass markers; A, B- PCR tubes coated with MAb 1/1, raised with serum core particles (IgM class); C, D- tubes coated with MAb ACAP27 (IgG class); E, F- tubes coated with irrelevant IgG control antibody; G H, tubes coated with irrelevant IgM MAb; J, K- tubes coated with PBS or BSA; I—positive control for RT-PCR, L—negative control for RT-PCR.

FIG. 13.

Analysis of the reactivity of human and mouse anti-core antibodies with recombinant core protein.

(A and B). Competitive binding assays with peroxidase-conjugated MAb ACAP27. Recombinant core protein (aa 1-110) was used as a solid-phase antigen. (A) A pool of globulins prepared from HCV-positive patients (HCIG), and normal human globulins (IgG) used as competitive antibodies and (B) unlabeled MAb ACAP27 and MAb VT (recognising different epitope, not overlapping with that recognised by MAb ACAP27) used as competing antibodies. (C). Analysis of the reactivity of HCIG and the ACAP27 and VT MAbs with recombinant core protein NC 360 by surface plasmon resonance.

FIG. 14.

Isolation of virus-like particles from insect cells infected with recombinant baculovirus containing e-DNA encoding the structural HCV proteins.

(A). Western blot analysis of the production of core protein in Sf9 cells, using MAb ACAP-27. Extracts, from recombinant baculovirus-infected cells (1) and (2) extracts from uninfected cells

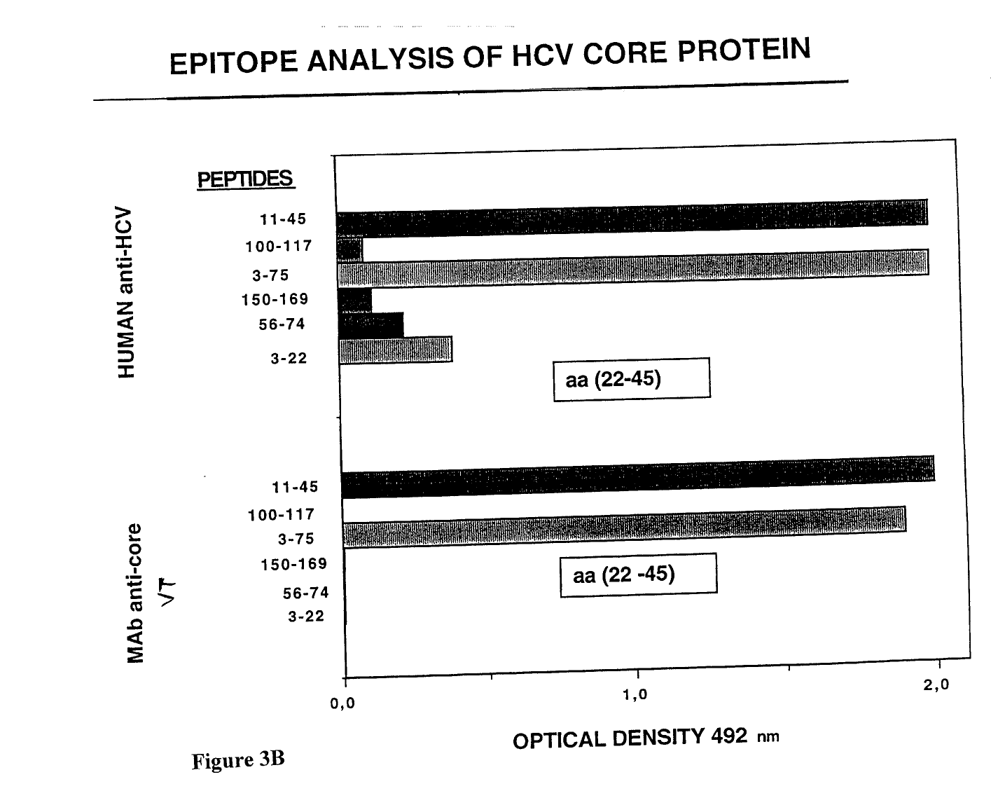

(B). Purification of nucleocapsid-like particles from lysed cells on a CsCl gradient. HCV core antigen was detected by ELISA and virus particles from the core antigen peak visualised by electron microscopy (FIG. 3 G and E).

FIG. 15.

Immunofluorescence staining of a liver specimen from a chimpanzee (CH 1572) experimentally inoculated with HCV, using the anti-core MAb 1/1, raised by immunisation with non-enveloped nucleocapsids purified from serum.

(A) Indirect immunostaining with MAb 1/1 followed by FITC-conjugated anti-mouse IgM, revealing a granular, fairly homogeneous pattern in the cytoplasm of hepatocytes. A group of four stained hepatocytes with one nucleus and two positive hepatocytes with two or more nuclei. displaying apple-green fluorescence.

(B) A negative control, CH 1572, before inoculation, immunostained with MAb 1/1, displaying rare weakly labelled sinusoidal granules. In both (A) and, especially, in (B) coarse granular, orange-yellow autofluoreseence of lipofuscin.

(C) Analysis of a liver section stained with MAb 1/1, examined by confocal microscopy, showing hepatocytes with granular or more homogeneous deposits binding MAb 1/1 (indirect immunostaining) as in (A), limited to the cytoplasm. The scale marker in (C) applies to all photographs (A-C).

FIG. 16.

Epitope mapping of the core antigen on circulating virus particles, using anti-core MAbs, and consensus within the sequence between aa (24-67) of the HCV core protein of various genotypes. MAbs VT and ACAP 27 were used in ELISA for detection of serum core particles and MAb 1/1 was generated by immunisation with HCV particles purified from plasma.

### DETAILED DESCRIPTION OF THE PREFERRED EMBODIMENTS

The present invention provides a surprisingly improved method for detecting HCV. In more detail, the present invention provides a method of detecting HCV by visualization of the presence of core protein by a double sandwich test using at least monoclonal antibodies produced by the hybridoma of the present invention.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and the various references cited therein. Furthermore, reference is made to standard protocols of producing antibodies, such as Harlow, Using Antibodies: A Laboratory Manual, Cold Spring Harbor Laboratory Press, Cold Spring Harbor, N.Y. (1999).

Hereinbelow is both a general and detailed description of the present invention involving the detection and immunological characterization of the free core antigen circulating in plasma of HCV carriers. Monoclonal antibodies were prepared by immunization of mice with a natural, serum-derived HCV nucleocapsid and applied for detection of HCV core in serum and liver tissue of HCV infected chimpanzees. 12. The method according to claim 11, wherein said second antibody is a mixture of at least two antibodies directed against the HCV core particle.

In the method of detecting the presence of HCV particles in a patient's plasma without chemical pretreatment one or more antibodies which recognize the nucleocapsid-like particle may be employed. In one embodiment one or more of the antibodies employed are selected from the group consisting of an antibody recognizing the amino acid region 24-37 of the core protein; an antibody recognizing the amino acid region 40-53 of the core protein; and an antibody recognizing the amino acid region 45-68 of the core protein.

Methods of labeling antibodies and detecting the label are known in the art and include labeling with one or more of the following enzymatically labeling, peroxidase, such as horseradish peroxidase, β-galactosidase alkaline phosphatase, radioactive marker, and/or fluorescent markers.

In the method of preparing a nucleocapside-like introducing the HCV-1 core gene can comprise any conventional method of introducing genes into a cell, e.g., calcium phosphate, electroporation, viral vectors, DEAE-Dextran, liposomes etc. Preferably the HCV-core gene is carried on a plasmid or contained within a viral vector. Examples of such viral vectors include baculovirus, adenovirus, retrovirus, adeno-associated virus, herpes virus, SV40, etc.

In one embodiment the introduction of the HCV-1 core gene comprises transfecting said eukaryotic cell with a plasmid carrying said HCV-l core gene. In another embodiment the introduction of the HCV-1 core gene comprises infecting said eukaryotic cell with a baculovirus carrying said HCV-1 core gene.

The invention further embodies nucleocapsid-like particle produced by the methods described herein as well as monoclonal or polyclonal antibody which binds to the nucleocapsid-like particles. Methods of preparing such antibodies can be performed using known methods, for example, as described in Harlow, Using Antibodies: A Laboratory Manual, Cold Spring Harbor Laboratory Press, Cold Spring Harbor, N.Y. (1999).

In another embodiment of the invention, detection kits for HCV in patients suspected of being infected with HCV, preferably HCV-1. In one embodiment the kit contains a negative and/or a positive control reagent.

### MATERIAL AND METHODS

### Measurement of HCV RNA by RT-PCR

HCV RNA in serum and fractions of the gradients was determined by nested polymerase chain reaction (PCR) based on the amplification of the cDNA from the core region of the virus. Viral RNA was isolated using a commercial Rnable reagent from Eurobio. cDNA synthesis and PCR was carried out with amplification using primers according to P. Simmons et al (J. Gen. Virol. 74,661-668, 1993) as detailed below:

RNA was reversely trans-transcribed using a primer sequence 5′-1.CATGTAAGGGTAATCGATGAC, cDNA was amplified using this primer and a primer in the 5′ NCR of the sequence 2. 5′-ACTGCCTGATAGGGTGCTTGCGAG. The second PCR used primers 3. AGGTCTCGTAGACCGTGCATCATG and 4. 5′-TTGCGGGACCTACGCCGGGGGTC.

### Detailed RT-PCT Method (RT-PCR Capsid-HCV)

This technique is described according to two protocols. The first one is a normal PCR amplification, but the usual dNTP mix is replaced with a dUTP/dATP, dCTP, dGTP mix in order to perform hydrolysis of the DNA with the Uracil-DNA Glycosylase (UDG) in the event of cross contamination of the PCR products. This hydrolysis step prior to PCR amplification is described in the second protocol. The principle of this hydrolysis step relies on the digestion of DNA matrices that contain dUTP instead of dATP, by the UDG. Only DNA which contains dUTP will be digested. However, precaution which must be taken in UDG use is to check the thermosensibility thereof. Indeed, after digestion of DNA matrix, it is necessary to inactivate the enzyme by heating during at least 5 min. at 95° C. to prevent digestion of the PCR products after amplification.

### Antibodies to HCV Core Protein and ELISA Test for Detection of HCV Core Protein

Monoclonal antibody VT directed to the HCV core protein was obtained from Valbiotech (Pads, France); MAb 39-72 was obtained by immunization of mice with a core peptide corresponding to the aminoacids sequence core numbers 39-72.

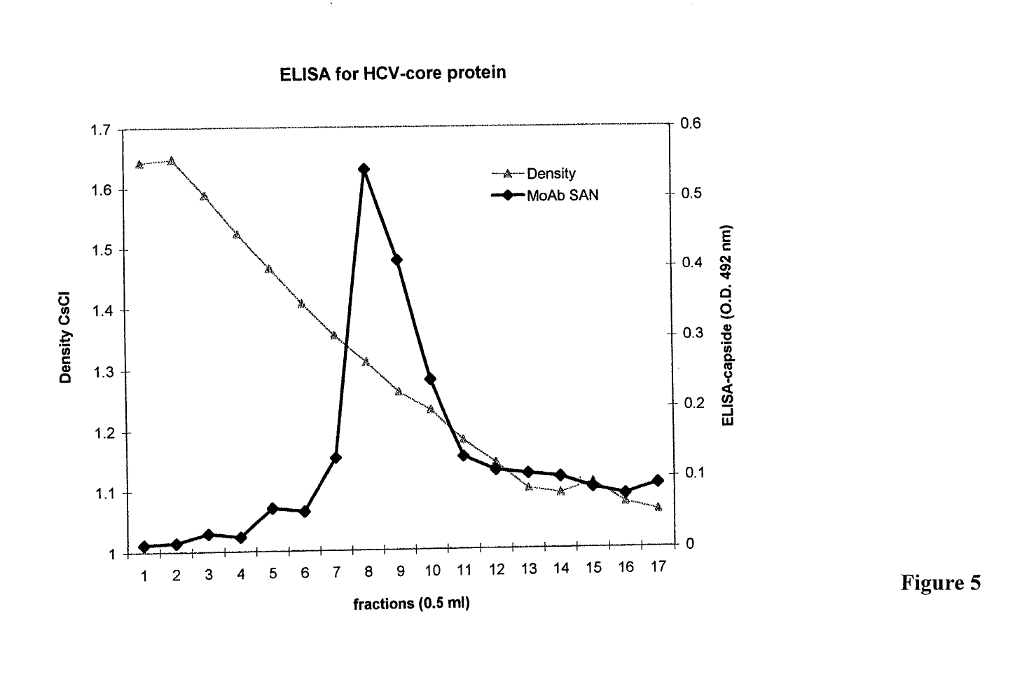

Analysis of the fractions of CsCl gradient with anti-core antibodies revealed the presence of the material reactive with both MAbs VT and 39-72 in fraction, defined as a “heavy fraction”, of a density of 1.32-1.36, much heavier than that of a presumed virion (1.08-1.10 g/ml) defined as the “light fraction”. This observation suggested the presence of non-enveloped nucleocapsids in plasma detectable by ELISA without any treatment. Moreover, the core epitopes detected were apparently exposed and non-covered by human immunoglobulins. Indeed, using inhibition assay no antibodies corresponding to MAb 39-72 could be were detected in a pool of human immunoglobulins prepared from sera with high titer of anti-core antibodies. See FIG. 5.

Two pools of human globulins containing anti-HCV antibodies IGIV and HG were prepared from sera of HCV carriers highly positive for anti-NS3, NS4 and core antibodies by Abbott HCV EIA were kindly provided by Dr. Ali Fattom, Nabi and A. Nowoslawski, respectively.

### Western Immunobloffing

Specimens were solubilized in a Tris buffer pH 6.8, containing either 2% SDS or 2% SDS and 5% 2-mercaptoethanol for 2 min at 100° C. Bromheriol blue (0.01%) and 20% sucrose were then added to the samples, the proteins were separated on 12% polyacrylamide gels, and electroblotted to nitrocellulose membranes. The membrane strips were postcoated overnight at 4° C. with 5% skim milk, washed and reacted for 1 h at room temp with monoclonal or polyclonal antibodies diluted in 1% skim milk. HRPO-labeled anti-mouse IgG (heavy+light chains) (Fab)′ fragments, Amersham) of anti-mouse IgM (Sigma) served as a second antibody. After final rinses the blots were visualized with an enhanced chemoluminescence detection system (Amersham).

### Epitope Mapping by ELISA

The wells of polyvinyl plates Maxisorb (Nunc, Denmark) were coated overnight at room temp, with 1 μg/ml of synthetic peptides corresponding to different arninoacid sequences of HCV core protein. The plates were washed with PBS containing 0.05% Tween and were blocked 2 h at 37° C. with 3% BSA in PBS containing 0.05% Tween 20. Monoclonal antibodies diluted in PBS were incubated on peptide-coated plated 2 h at 37° C. Following washing as above the wells were incubated with HRPO-labeled anti-mouse IgG (heavy+light chains) (Fab)′ fragments, Amersham) of anti- mouse IgM (Sigma) as a second antibody. The reaction was developed using o-phenylenediamine as the enzyme substrate and the absorbance values were read at 492 nm with an ELISA plates reader.

### Competition ELISA with Human and MAbs Anti-HCV Core

Polyvinyl plates were coated with synthetic peptides or purified recombinant proteins in a concentration of 1 Fg/ml, blocked and washed as described above. 100 microliters of human globulins prepared from human sera with a high titers of anti-core antibodies or MAbs, fluids were added to the wells and incubated 24 h at 37° C. After washing, peroxidase labeled MAb 39-72 was added to the wells and incubated as before. The plates were developed and read as described above.

### Preparation of Mabs to HCV Core Protein

Balb/c mice were immunized intrasplenically with 50 μl of the fraction of CsCl gradient containing HCV core antigen detectable by ELISA. The fraction was dialyzed against PBS and concentrated using Nanosep centrifugal concentrator 300K (Pall Filtron). Three days after immunization mice spleen cells were fused with Sp2/OAg-myeloma cell line. Hybridoma supernatants were screen by ELISA using purified recombinant core protein corresponding to amino acids no. 1 to 120 of the sequence of the nucleocapside. The hybridomas reactive with the recombinant protein were cloned by limiting dilution. The immunoglobulin class of MAbs was determined using anti-mouse IgG (g chain) Amersham and anti-mouse IgM (m chain) (Sigma). The epitope specificity of MAbs was determined using a series of synthetic core peptides.

### RESULTS

### Fractionation of HCV in Density Gradients

Precipitation of HCV by PEG-6000, previously used for concentration of other viruses allowed the concentration of HCV without lost of viral RNA. Notably, the totality of HCV RNA present originally in the plasma was recovered in the pellet. PEG precipitated preparation was subsequently submitted to ultracentrifugation in sucrose or CsCI gradients. Analysis of distribution of HCV by PCR in sucrose and CsCl gradients after equilibrium centrifugation showed heterogeneity of viral material derived from plasma. The majority of viral RNA was detected in top fractions of a buoyant density of 1.08-1.10 g/ml CsCl and at the density of 1.08 g/ml of sucrose. This RNA could be probably attributed to the b- lipoprotein associated virions since in RNA present in the “light” (top) fractions was stable and could be precipitated 90%with both dextrin sulphate.

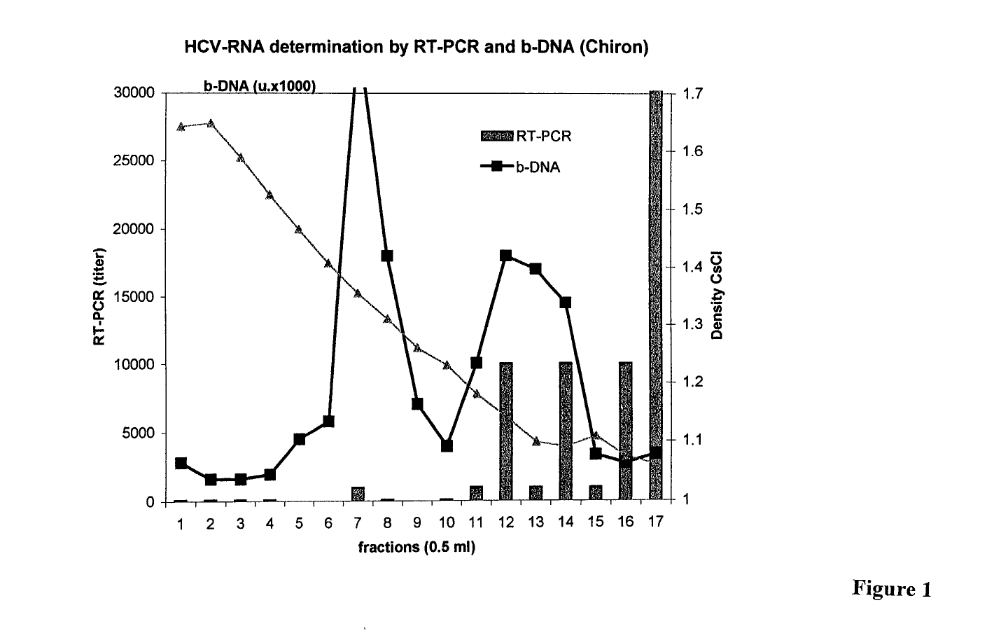

A part of viral RNA was localized in fractions of higher density. See FIG. 1. Interestingly, different profiles of the distribution of viral RNA in the gradient were obtained using routine PCR and a commercial b-DNA assay (Chiron) which apparently does not detect he bulk of viral RNA at the top of the gradient.

### Fractionation of HCV Positive Human Plasma

Human plasma (100 ml) from a chronic HCV carrier (voluntary blood donor) seropositive for anti-HCV antibodies and containing HCV of 1 a genotype (titre 10-5 by PCR) was stored at 80° C. The plasma was thawed and clarified 10 min at 10,000 rpm, PEG 6000 was then added to the clarified plasma to a final concentration of 10% and NaCl to a final concentration of 0.4 M. The mixture was incubated overnight at 4° C. and precipitated virus separated by centrifugation for 1 h at 11,000 rpm in rotor of a Centrikon centrifuge. The pellet, was resuspended in a 13 ml of a 0.01 M Tris-HCl pH 7.2 containing 0.15 M NaCl. The pellet was subjected to centrifugation in a discontinuous CsCl gradient (1.10-1.60; g/ml 1.5 ml of each solution) prepared in PBS and containing protease inhibitors- I mM PMSF, 2 pg/ml a protein and 10 mM EDTA. Centrifugation was carried out in a Beckman SW 41 rotor 48 h at 40,000 rpm. Fractions (1 ml) were collected from the bottom of the tube and assayed for HCV RNA by PCR and for the presence of core antigen by ELISA.

### Detection of HCV Core Antigen in Fractions of CsCl Gradient by ELISA

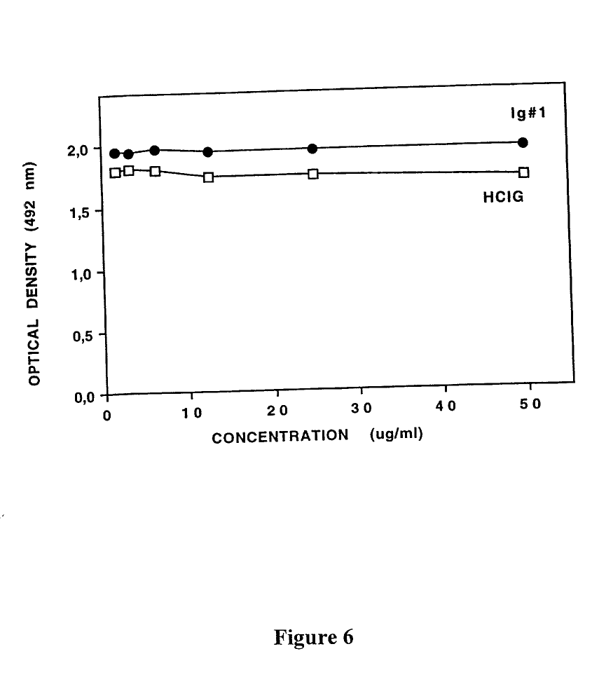

Two MAbs, designated as MAb VT (Valbiotech, Paris, France, immunizing antigen non-communicated by the producer) and MAb 39-72, obtained by immunization of mice with a core peptide corresponding to the aminoacids sequence core numbers 39-72 were used for the development of the assay for detection of the HCV core protein. See FIGS. 2A and 2B. The specificity of these monoclonal antibodies was ascertained by Western blot with recombinant HCV core proteins and epitopes recognized by these MAbs were delineated using a series of synthetic peptides encompassing HCV core protein: MAb VT was reactive with the epitope located in the aminoacid sequence 24-37, and MAb 39-72 was reactive with an epitope located in the aa sequence 40-54. See FIGS. 3A-3B. The competitive binding assay confirmed that these two MAbs recognized two different, non-overlapping and non- adjacent epitopes. See FIG. 6.

To exclude the possibility of the interference of rheumatoid factor (RF) or other non- specific reactivity with the detection of the core antigen, the presence of RF in the gradient was tested. The RF reactivity was detected by latex test in parallel with the non-specific binding to a control (unrelated to HCV) in the fractions of the gradient located at the lower density than that of the core activity.

To evidence that, in fact, core antigen was present in the gradient, fractions exhibiting the core antigenicity were polled, concentrated by dialysis in the Nanosep centrifugal concentrator 300K (Pall Filtron). 300,000 kda and injected to Balb/c mice to produce MAbs. The hybridomas were selected by ELISA with synthetic 1-130 peptide and subsequently tested with a series of overlapping peptides corresponding to different regions of the core antigen. According to these results, it was deduced that the obtained MAb recognized a linear epitope which is localized in the aminoacid sequence (45-75) of the core region.

The development of an effective vaccine against HCV is important, but is rendered difficult because of the variability of the virus and unknown antigenic structure of the virion. Identification of the epitopes conserved among different HCV genotypes would be of importance for future development of the immunological assays for detection of the HCV proteins in serum.

The physical properties of HCV particles have been analyzed by ultracentrifugation in sucrose gradients by several groups. Two main populations of HCV particles according to their floating density were found in sera of patients with chronic HCV infection: low-density virus particles (1.06-1.12 g/ml) and high density virus particles (1. 18-1.21 g/ml). Virus particles with high density has been apparently associated with immunoglobulins or was supposed to represent partially or completely naked nucleocapsids Kanto. The virus particles of low density were not associated with immunoglobulins, and accumulated base changes in the hyper variable region of the E2 envelope domain of the genome. Changes in the relative proportions of these viral populations have been observed. Kanto and Hino. The increase of the relative numbers of the high density virions correlated with the disease activity and heterogeneity in HVR1 region, whereas patients with a predominance of the low density fraction showed sustained response to interferon treatment.

Core antigen has been detected in by use of monoclonal antibodies after treatment of serum concentrates with detergents or denaturing agents. Tak, Tanaka, Kashiwakuma, Orito, and Takahashi. The core antigen was detected in sera of non-responders to IFN-a but not in patients with a sustained response and was correlated to the level of viral RNA. Tanaka. Isolated nucleocapsid-like HCV particles were observed by electron-microscopy (EM) of the detergent-treated, RNA rich fractions. Taka. Few reports suggested the presence of naked, unenveloped HCV nucleocapsids in sera of HCV carriers which could be observed by EM Trest, or detected in serum by Mabs. Kanto and Maslowa. However this population of HCV has not yet been isolated and characterized immunochemically.

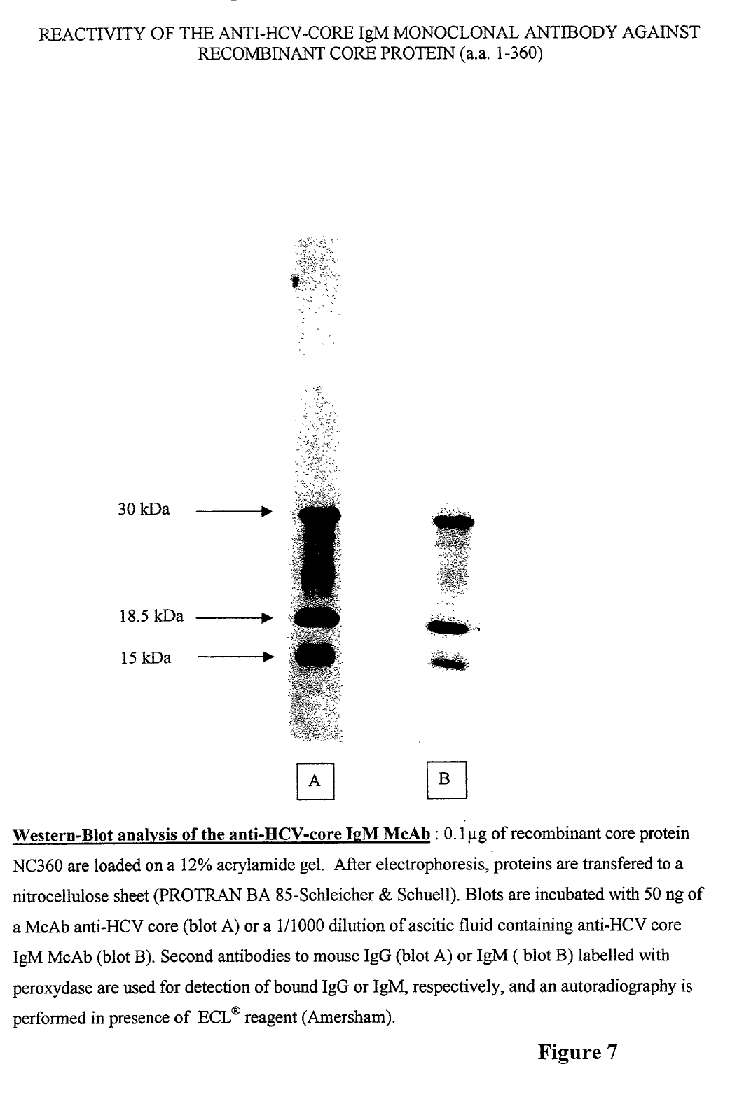

In the following experiment using well-characterized MAbs, the core epitopes exposed on the native nucleocapsid protein were detected in serum. These monoclonal antibodies recognized the non-overlapping epitopes of the HCV core, located close to each other in the aminoacid sequence 24-53. Since reactive with MAbs, these epitopes were not covered by human anti-core antibodies and no corresponding specificity could be detected in a pool of antibodies from chronic HCV carriers. The core antigen was isolated from serum and was shown to be immunogenic in mice. MAbs obtained by immunization with a native serum-derived core protein bound to the linear epitope located in the aa sequence (45-68) as evidenced with synthetic peptides and recognized recombinant cone protein in Western blot. See FIG. 7. This epitope is conserved between different HCV genotypes and is adjacent to the epitopes recognized by the MAb 39-72 used for detection of the core antigen in plasma.

MAbs raised against the natural core antigen was used to detect HCV core antigen in a liver tissue of chronically infected chimpanzee. This MAb represents a new reagent for the study of HCV biology and for the immunological detection of the viral antigen in sera of patients with HCV infection.

### PROTOCOLS USED

### I-RNA Extraction from Serum

In a 1.5 ml Eppendorf tube, extract 100 μl, 10 μl and 1 μl of each serum sample. Add respectively 0 μl, 90 μl or 99 μl of sterile water (qsp 100 μl).

Add 1 ml of RNable® (Eurobio). Mix 20 sec. and let 5 min. on ice.

Add 100 μl (1/10th vol.) CHCl₃ (ReadyRed, Appligene), mix and centrifuge 10 min. at 14000 rpm. Save the colorless supernatant in a new tube.

Add 500 μl of CHCl₃, mix and centrifuge 10 min.

Save the supernatant (#500 μl) and add 50 μl 3M NaOAc pH 5.2, 2 μl of See DNA™ (Amersham, RPN 5200) and proceed to an ethanol precipitation with 2 vol. (1 ml) of 100% ethanol. Mix, and centrifuge 10 min. at 1400 rpm at 4° C.

Wash the RNA pellet with 1 ml of cold 70% ethanol. Centrifuge 10 min.

Remove all the supernatant, dry the walls of the tube with a Kimwipes® and resuspend the RNA pellet with 20 μl of water containing 2 mM DTT and 2 U/μl Rnasin.

Store at −80° C.

### I-cDNA Synthesis (Common to Both Protocols).

In a 500 μl Eppendorf tube, add: (final conc.)

5 μl of purified RNA

5 μl of water

and 1 μl reverse-sense primer SIM 2R.

Recover the mix with one drop of mineral oil, centrifuge briefly and place on the thermocycler for 10 min. at 70° C. and immediately on ice. Centrifuge before the addition of 14 μl of the following mix:

5 μl reverse-transcription buffer 5× (1×)

1.25 μl dNTP 10 mM (0.5 mM)

0.5 μl DTT 100 mM (2 mM)

1 μl RNase inhibitor 40 U/μl (1.6 U/μl)

1.25 MMLV 200U/μl (250 U)

5 μl H₂O (qsp 25 μl)

Centrifuge briefly before incubation 1 hour at 37° C. Inactivate the RT during 10 min. at 95° C. and dip the tubes on ice. At this step, the cDNA can be kept at −80° C.

II-PCR amplification

| A1 - Outer PCR | A2 - Outer PCR |
|---|---|
| without UDG hydrolysis | with UDG hydrolysis |
| Prepare a mix of these components | Prepare a mix of these components |
| in a 1 ml Eppendorf tube on ice | in a 1 ml Eppendorf tube on ice |
| (final conc.): | (final conc.): |
| 5 μl buffer 10 X | 5 μl buffer 10 X |
| 1.5 μl MgCl₂ 50 mM (1.5 mM) | 1.5 μl MgCl₂ 50 mM (1.5 mM) |
| 2.5 μl dUTP/NTP mix 4 mM | 2.5 μl dUTP/NTP mix 4 mM |
| (0.2 mM) | (0.2 mM) |
| 1 μl reverse sense primer SIM | 1 μl sense primer SIM 1S |
| 2R (50 pmole) | (50 pmole) |
| 33.5 μl H₂O (qsp 50 μl) | 1 μl reverse sense primer SIM |
| 0.5 μl Eurobiotaq (2.5 U) | 2R (50 pmole) |
| | 32.5 μl H₂O (qsp 50 μl) |
| | 0.5 μl UDG (0.5 U) |
| | 0.5 μl Eurobiotaq (2.5 U) |

Dispense 45 μl of this mix in each thin-walled PCR tube on ice. Add a drop of mineral oil and close the caps.

Under the hood: add 5 μl of cDNA, centrifuge the tubes briefly and put them:

in the thermocycler block once the temperature has reached at least 80° C. (Program No. 6)

at 37° C. during 15 min. and then, denature the UDG during 5 min. at 85° C. and 10 min. at 95° C. before starting the amplification (Program No. 5)

| Amplification cycles (Prog. 6): | | Amplification cycles (Prog. 5): | |
|---|---|---|---|
| First Cycle: | 94° C.-5 min. | First Cycle: | 85° C.-5 min. |
| | 50° C.-1 min. | | 95° C.-10 min. |
| | 72° C.-1 min. | | 50° C.-1 min. |
| | | | 72° C.-1 min. |
| 25 cycles: | 94° C.-50″ | 25 cycles: | 94° C.-50″ |
| | 55° C.-50″ | | 55° C.-50″ |
| | 72° C.-50″ | | 72° C.-50″ |
| Elongation: | 72° C.-10 min. | Elongation: | 72° C.-10 min. |
| Stop: | 4° C.-5 min. | Stop: | 4° C.-5 min. |

Note:
At the end of this application it is important to centrifuge the tubes before opening to avoid contamination and to perform the nested amplification immediately.

B—Inner PCR

This step is common to both protocol because the first amplification product must not be digested by UDG.

Prepare a mix of these components in a 1 ml Eppendorf tube on ice:

5 μl buffer 10×

1.5 μl MgCl₂ 50 mM (1.25 mM)

2.5 μl dUTP/NTP mix 4 mM (0.2 mM)

1 μl internal sense primer SIM 3S (50 pmole)

1 μl internal reverse sense primer SIM 4R (50 pmole)

33.5 μl H₂O (qsp 50 μl)

0.5 μl Eurobiotaq (2.5 U)

Dispense 45 μl of this mix in each thin-walled 0.5 ml PCR tubes on ice. Add a drop of mineral oil.

Under the hood: add 5 μl of DNA, centrifuge and put the tubes on the PCR block once the temperature has reached at least 80° C.

| | | |
|---|---|---|
| Amplification cycles: | First cycle: | 94° C.-5 min. |
| (Program No. 6) | | 50° C.-1 min. |
| | | 72° C.-1 min. |
| | 25 Cycles: | 94° C.-50″ |
| | | 55° C.-50″ |
| | | 72° C.-50″ |
| | Elongation: | 72° C.-10 min. |
| | Stop: | 4° C.-5 min. |

Note:
At the end of the amplification it is important to centrifuge the tubes before opening to avoid contaminations and to analyze the products immediately or maintain them at −20° C.

Preparation of dUTP/dNTP-mix

Stock solutions:

250 μl-20 mM solution dUTP (Epicentre/TEBU)

25 μM-100 mM dUTP solution (USB/Amersham)

dNTP 25 μM-100 mM solutions kit Pharmacia

Preparation:

A-TEBU 20 mM dUTP:

Dilute 1/25 the 100 mM solutions of the dATP, dCTP and cGTP (4 mM final)

Dilute the Epicentre/TEBU dUTP 20 mM solution ¼ to get a 5 mM solution.

Mix 1 vol. of each dNTP diluted solution to get the 4 mM solution of dUTP/dNTP mixture.

B-USB 100 mM dUTP:

In a 1.5 ml tube, add 40 μl of each dATP, dCTP and dGTP 100 mM stock solutions (Pharmacia) and 50 l of the 100 mM dUTP stock solution (USB). Complete to 1 ml (830 μl) with sterile water to obtain the 4 mM solution of dUTP/dNTP mixture.

Preparation of the Solution to Resuspend RNA Pellets

930 μl pure sterilized water

20 μl 0.1 M DTT

50 μl Rnasin

### Analysis of the Distribution of HCV RNA by RT-PCR and B-DNA in CsCl Gradient

The majority of viral RNA was detected by RT-PCR in top fraction (“light fraction”) of the gradient corresponding to buoyant density of 1.06-1 .10 g/ml CsCl. According to the literature (and also our observation that the majority HCV RNA detectable by RT-PCR can be precipitated with dextran sulfate) this part of RNA could be attributed to the HCV virions associated to β-lipoproteins.

Only a minor part of viral RNA was detected by RT-PCR in fractions of higher density; in contrast b-DNA assay which was much more effective at higher density range and two peaks of HCV RNA could be detected by this assay at 132-36 and the second at 1 .10- 1.15 g/ml. Moreover, the peak of RNA detected by b-DNA at the density of 1.32-1.36 g/ml corresponded to the localization of the core antigen by ELISA.

The hybridoma described in the present application was deposited at the C.N. C.M. in France on April 14, 1999, under accession number 1-2183.

Having described the present invention, it will now be apparent that many changes and modifications may be made to the above-described embodiments without departing from the spirit and the scope of the present invention.

Serum and plasma samples eruzn and plasma sarnpics Plasma and serum samples were obtained from volunteer blood donors with normal alanine transaminase (ALT) levels who tested positive for anti-HCV antibodies by MONOLISA anti-HCV PLUS (BIO-Rad, Marnes la Conquette,France)and RIBA(ORTHO Diagnostics) and for HCV RNA by RT-PCR.Plasma samples were stored frozen at −80° C.. Serum samples, obtained from chronic HCV carriers testing positive for IICV markers in routine assays as described above, were analysed, without freezing, a few hours after blood sampling.

Serum samples from 2 chimpanzees experimentally infected with HCV were tested for the presence of circulating core antigen on day 0, 28g and 45 after inoculation for chimpanzee (CH) 1537 and on day 0, 38 and 52 for CH1572. ALT levels raised above cut off values on day 13 after inoculation in C11 537 and on day 7 in CH 1572 and reached peak values on day 77 and 74 after inoculation, for CH 1537 and CH 1572, respectively. Both chimpanzees tested positive for HCV RNA (5.8 logs international units (IU)3ml in CH 1537, 5.7 logs 1IU/ml in CH 1572 by Amplicor Monitor, Rochc). All serum samples from CH 1537 and CH 1572 were negative for anti-HCV core antibodies (RIBA HCV 3.0, Ortho Diagnostics). A sample from CH572, obtained 45 days after inoculation was positive for anti--NS4(ci 00) and NS3 (c33e3).

Recombinant core proteins

Recombinant HCV core proteins produced in HelpG2 cells were isolated as previously described (8, 11 ). Purified recombinant HCV core protein NC 360 (ac 1-120), produced in F,. COi, was kindly provided by J. F. Delagneau, (.Bio-Rad Labs, Marne la Coquette, France).

Monoclonal and polyclonal anti-HCV antibodies

The anti-core VT Mab from Valbiotech (Paris, France, immunogen non-communicated). Mab ACAP27 was obtained by immunisation of mice with a synthetic peptide corresponding to the aa sequence (39-72) of the HCV core protein and was provided by J.F. Delangnean MAB 1/1 was produced in the study by immunisation of mice with a synthetic peptide nucleocapsids isolated from plasma of asymptomatic HCV carriers as described below. This antibody recognises aa sequence (45-68) of thc core protein.

The anti-E1 (A4) and anti-E2 (A11) MABs been described elsewhere (8,11) Globtilin fraction (HCIG), prepared from the sera of HCV carriers strongly positive for anti-core, anti-NS3. and anti-N4 antibodies in Abbott HCV HIA, was kindly provided by Ali Fattomi (Nabi, Rockville,MD,USA). Normal human globulins were obtained from Sigma.

Fractionation of HCV-positive plasma

Plasma samples (RT-PCR. titre 10₅-10₇,genotype 1aor 1b) were thawed and clarified by 0 centrifugation for 10min at 20,000×g. Virus particles were precipitated overnight at 4° C.in 10% PEG 6000 supplemented with 0.4 M NaCl. The precipitate was collected by centrifugation for 1h at 14,000 rpm in the SW-41 rotor of Reckman centrifuge. The pellet, containing all the HCV-RNA initially detected in plasma, was resuspended in 7.5 ml of 0.01 M Tris-HCl pH 7.2, 0.15 M NaCl and 10 mM EDTA. It was then subjected to centrifugation on a discontinuous CsCI density gradient (1.10- 1.60 g/ml) in a Beckman SW 41 rotor at 40,000 rpm for 48h at 4° C. Fractions (0.7ml), were collected from the bottom of tie tube and were assayed for HCV RNA by RT-PCR and for the presence of HCV core antigen by ELISA. All reagents used in the purification procedure contained protease inhibitors: 1mM PMSF, 2 μg/ml aprotinin and 10 mM EDTA. FLtISA for the detection of IICV core antigen Polyvinyl plates (Maxisorb, Nuric, Denmark) were coated with the VT or ACAP-27 MAb, at a concentration of 5 μg/ml. They were then saturated with 3% BSAt, (.05% Tweeni-20 hi PBST and incubated with I 00 put of the sample to be tested for the presence of core anti the hound antigen was detected with the horseradish peroxidase (HRPPO) consulted iMiAb AC,AP27. using orthophenylcncdiarnine (OPD) as the substrate. Absorbance at 479 im. was determined with a TittCk ifulntiscan QLISA plate reader. Preparation of noncolonial antibodies against serum-(teried core antigens Fractions obtained after centifusion of HCV positive plasma. ii the CsCI gradient and containing core particles were pooled, dialysed against PBS and concentrated using a Nianosep Cetntrifiual Concentrator 300K. (Pall Filtron, St. Ucrcnaiii en l.₁aye. TFrance). Aliquots (50pull) of tie preparation were injected into the spiceni of Trjalh/c mice. Tiicc days after immuaisatiuin, mouse spleen cells were filled with cells of the Sp2lloAg- myeloma cell line. Hybridoma supernatants were screened by ELISA, using the purified recombinant core protein N(′ 3)60 aa (2-120) and a positive hybridoma cloned by limiting dilution. The immunoglobulin class of MAbs was determined using anti-mouse IgC 7-cllain (iktncrshain) and anti-mouse igM w-clhain (Sigrma) antibodies.

Epitope mapping of MiAbs with synthetic core peptides

The epitope specificity of anti-core NIAbs (ACAP 27, VT and MAb 1/1) and human anti- FHCV globulins (HCIG) was detemined using a portion of synthetic peptikes corresponding to fragments of HCV core protein. Syintlietic core peptides were kindly provided by A. Kolubov and J. F. Delagneau. ELISA was carried out as described above, using core peptides at a concentration of 1 μg/ml to coat the plates. Bound antibodies were detected with HRPO-conjugated anti-mouse IgC, (heavy+light chains) (Fab)2 fragments (Amersham), anti-mouse IgM (Sigma) or anti-human IgG (Dako).

Competitive inhibition assays Competitive inhibition assays were carried out to investigate the epitope specificity of anti-core MAbs and to analyse the capacity of human anti-core anitibodies to inhibit the reactivity of MAbs with HCV core protein. For these assays polyvinyl plates were coated with purified recombinant HCV core protein NC 360 (aa1-120), at a concentration of 1 μg/ml. The plates were blocked and washed as described above. One hundred microliters of human anti-HCV globulins (HCIG) or normal human gobulins, as a control, or unlabeled MAbs, diluted in PBS were added to the wells and incubated for 2 h at 37° C. The plates were washed; and peroxidase-conjugated anti-core MAb (ACAP 27 or MAb 1/l) was added and the plates incubated for 1 h at 37° C. The plates were developed and read as described above.

RT-PCR for determination of HCV RNA

HCV RNA was determined by nested polymerase chain reaction (PCR), based on amplification of the cDNA from the core region of the viral genome. For RT-PCR, viral RNA was extracted using the commercial RNable reagent (Eurobio, Les Ulis, France). RNA was reverse-transcribed using the primer 5′-CAT/GGTA/GAGGGTATCGATGAC-3′. The cDNA was amplified using this primer and a primer binding to the 5′ non-coding region: 5′- ACTGCCTGATAGGGTGCTTGCGAG-3′. Nested PCR was performed with the primers: 5′- AGGTCTCGTAGACCGTGCATCATG-3′ and 5′- TTGCGG/TG/CACCTA/TCGCCGGGGGTC-3′.

Affinity-.capture RT-PCR

Affinity-capture-RT-PCR was carried out as described by Han et al. (12). PCR tubes were coated by incubation overnight with 50 μg/ml of anti-core MAb ACAP 27 (IgG1) or MAb 1/l(IgM) or control MAbs nor-related to HCV of IgC1 and IgM class, or with 1 % BSA in PBS. Serum samples (50 μl ) from patient testing positive for HCV infection in serological tests were then incubated in antibody-coated or control tubes. RNA was then extracted from the adsorbed material and used for RT-PCR, as described above.

Isolation of nucleocapsid-like particles from recombinant baculovirus-infected insect cells

Nucleocapsid-like particles were isolated from Sf9 (Spodoptera frugiperda) cells infected with recobinant baculovirus, according to the procedure described by Baumert et al (3,4). A recombinant baculovirus containing a cDNA encoding the structural proteins of HCV was kindly provided by J. Liang, NIH, Bethedsa. Insect cells were lysed as previously described (3,4). Cell lysates were concentrated by precipitation overnight at 4° C. in 4% PHG, 0.4 M NaCl and were then layered onto a discontinuous 1.1-1.6 g/ml CsCl gradient and centrifuged for 24 h at 4° C. at 41,000 rpm in the SW 41 rotor of a Beckman ultracentrifuge. Fractions (0.5 ml) were tested for HCV antigens by ELISA. All reagents used for the purification of nucleocapsid-like particles e contained protease inhibitor cocktail (Boehringer, Mannheim).

Surface plasmon resonance (SPR) analysis

Surface plasmon resonance analysis was carried out with a Biacore^{R} 2000) (Biacore AB, Uppsala, Sweden). All reagents, including the P20 surfactant, the amine-coupling kit containing N-hydroxy-succinimide and N-ethyl-N4-(3-diethylaminorpropyl) carbodiimide, ethanolamaine hydrochloide (EDC(NHS 1/l) and CM-5 sensor chips were obtained from Biacore. The running and dilution buffer (IIBS-EP, pH 7.4) consisted of 10 mM Hepes 150 mM NaCl, 3.4 mM EDTA and 0.005P2O surfactant.

Recombinant core protein NC 360 (50μg/ml in phosphate buffer pH 7.2) was covalently coupled via primary amino groups to the CM-5 sensor chip, using the amine-coupling procedure. The SPR signals for NC-360 protein were 4500 resonance units (RU) where 1 RU corresponds to an immobilised protein concentration of 1 pg/mm². Monoclonal anti-core antibodies (1-20 μg/ml) or human globulins (in concentrations 20 to 200 μg/ml) or control antibodies unrelated to HCV (in corresponding concentrations) were injected in running buffer. Changes in surface concentration resulting from interaction of the antibody with surface-fixed antigen were detected as an optical phenomenon affecting the surface plasmon resonance signal, expressed in resonance units. Kinetic constants, association rate constants and dissociation rate constants were calculated with BIAEVALUATION 3.1 software.

Affinity chromatography

MAb ACAP27 was purified from ascitic fluid by chromatography, using a Hitrap protein A column (Amersham, Pharmacia Biotech). The MAb (5.3 mg) was bound to the Affigel Hz (BioRad) as recommended by the manufacturer. A serum sample (35ml) from a chronic HCV carrier testing positive for HCV core antigen by ELISA was applied to the column, which was then incubated for 2 h at room temperature. The column was thoroughly washed with PBS and bound antigen was eluted from the column with 200 mM. glycine-HCl buffer pH 2.5. The pH of the eluate was immediately adjusted to neutral with 1M Tris. The eluate was then tested for core antigen by ELISA, for IICV-RNA by PCR and for virus particles by electron microscopy.

Electron microscopy.

Formvar-coated microscope grids (200 or 300 mesh) were incubated with fractions of the gradient diluted 1:10 in PBS. Virus particles were stained with 1% urananyl acetate in distilled water and the grids were observed in a Phillips CM-10 electron microscopic. For solid-phase immune-electron -microscopy, -formvar-coated grids were first incubated for 5 min with anti-core, (MAb ACAP 27 or MAb 1/I) anti-E2(MAb A1l), anti-FI (MAb A4), or control MAbs (non related to HCV) at a concentration of 5μg/ml in Tris-HCl pH.8.0. They were then washed with the same buffer. A drop of the preparation containing virus particles was then placed on the grid without drying and incubated for 10 min. Grids were then washed with PBS, stained with 1% uranyl acetate and examined as described above.

Western immunoblotting

Samples were solubilized by incubation in Tris pH 6.8, 2% SDS and 5% 2-mercaptoethaniol for 2 min at 100° C. They were then subjected to electrophoresis in 12% polyacrylamide gels and electroblotted onto nitrocellulose membranes. Membrane strips were incubated overnight at 4° C. with 5% skimmed milk powder and 0.1% Tween 20 in PBS, washed and incubated for 1 h at 37° C. with anti-core MAbs (ACAP 27 or VT) or E1 (A4) and E2 (A1l) diluted in 1% skimmed milk powder. HRPO-conjugated anti-mouse IgG (heavy+light chains) (Fab)2 fragments (Amersham). Blots were rinsed and developed with an enhanced chemoluminescence detection system (Amersham. Little Chalfont United Kingdom).

Immunofluorescence staining of HCV-infected liver tissue.

The experimental protocols for HCV-infected chimpanzees, including details of animal care and housing were approved by the Centers for Disease Control and Prevention institutional Animal Care and Use Committee. Surgical liver biopsy specimens were obtained from two chimpanzees (CH 1572 and CH1537) infected with HCV gentype 1a (CDC/ Chiron US-1 strain) (6), 36 and 42 days after inoculation respectively. In the specimens, 50 to 70% of hepatocytes contained HCV antigens, as assessed by staining, with fluorescent isothiocyanate (FITC)-conjugated polyclonal IgG fractions from the sera of individuals with chronic HCV infection (21). Negative controls included biopsy specimens taken from Cl11572 and (CH1537 before inoculation with HCV, specimens from two uninfected, naive chimpanzees, and specimens from chimpanzees infected with either HAV or HBV.

Cryostat sections (5-8 μm thick) were fixed in anesthetic either for 5 min, air dried, and incubated for 1 hour at room temperature with MAb1/l pre-absorbed onto a liver homogenate prepared from a naive chimpanzee. Section were washed in PBS and incubated with FlTC-conjugated goat F(ab)′2 fragment against mouse IgM (μ chain) (Cappel, ICN Pharmaceuticals, Aurora, Ohio), at concentration of 10 μg/ml, to detect bound MAb1/l. The slides were examined with a Zeiss microscope equipped with an epifluorescence device and a HBO 100/W2 illuminator. Controls included stainings of cryostat sections of HCV-infected livers from CII1572 and CH 1537 with the FITC-conjugated anti-mouse IgM antibody, and stainings of these sections with PBS or an irrelevant mouse MAb of the IgM class in the place of primary antibody. Sections from HCV-infected CH1572 and CHH1537 livers stained with MAb1/1 were examined with a confocal laser microscope (Zeiss LSM 510).

### RESULTS

ELISA for detection of the HCV core antigen

Specificity of MAbs used to detect HCV core antigen was ascertained by Western blotting, using recombinant core protein produced in HepG2 cells. These MAbs reacted with protein bands corresponding to the two previously described forms (36) of the core protein; p23 and p21 (data not shown). The epitopes recognised by these MAbs were determined using a panel of synthetic peptides covering the HCV core protein: MAB VT reacted with aa sequence (24-37) and MAb ACAP27 reacted with aa sequence (40-53) (FIG. 1). These MABs had extremely high affinity constants, as determined by SPR analysis (see below). The detection threshold of ELISA with either of these MAbs on the solid phase and peroxidase-conjugated MAb ACAP 27 was about 1 ng, as determined using the recombinant NC 360 core protein (aa 1-110) as reference antigen.

Isolation of HCV core particles from plasma

Analysis of the fractions collected after equilibrium centrifugation of HCV-positive plasma showed that the major peak of viral RNA (titer 10⁵, determined by RT-PCR) occurred at a density of 1.06-1.18 g/ml, corresponding to the putative, β-lipoprotein-associated virions (41). Direct ELISA revealed the presence of HCV core antigen in a large peak at a density of 1.27-1.35 g/ml (FIG. 2A), in fractions containing 100-1000 times less of HCV-RNA (titer 10², as determined by RT-PCR) than fractions containing putative virions. Core antigen-positive fractions, subjected to a second centrifugation in the same conditions, banded at a density of 1.32-1.34 g/ml (not shown) Virus particles, heterogeneous in size, with predominant populations of 38-43 nm and 54-62 nm in diameter were observed in these fractions by electron microscopy (FIG. 3 A, B). These viral particles were bound to microscope grids by anti-core MAbs (MAb ACAP 27 or MAb 1/1) but not by anti-E1 and anti-E2 or control MAbs. The relative proportions of these particles differed between HCV preparations and no particles in aggregates were observed.

Isolation of HCV nucleocapsids from virions

To compare the properties of HCV core particles naturally occurring in serum with HCV nucleocapsids isolated from putative HCV virions, an aliquot (1.5 ml) of a fraction corresponding to the HCV-RNA peak (density of 1.10 g/ml) was treated with 0.5% Tween-80, and subjected to centrifugation in a CsCl gradient. HCV core antigen appeared at a density of 1.32-34 g/ml, accompanied by a shift of HCV RNA from the light region of the gradient (FIG. 2 B). Virus particles, mostly 38-43 nm in diameter but also larger particles of 54-62 nm were observed in these fractions by electron microscopy (FIG. 3D, E, F). Both types of particles were bound to microscope grids by anti-core antibodies. This experiment showed that the HCV particles occurring naturally in the plasma of chronically infected patients and expressing core antigen at their surface, had buoyant density, morphological and antigenic properties similar to those of HCV nucleocapsids released from virions by detergent treatment.

Production of new MAbs by immunisation with core particles from serum

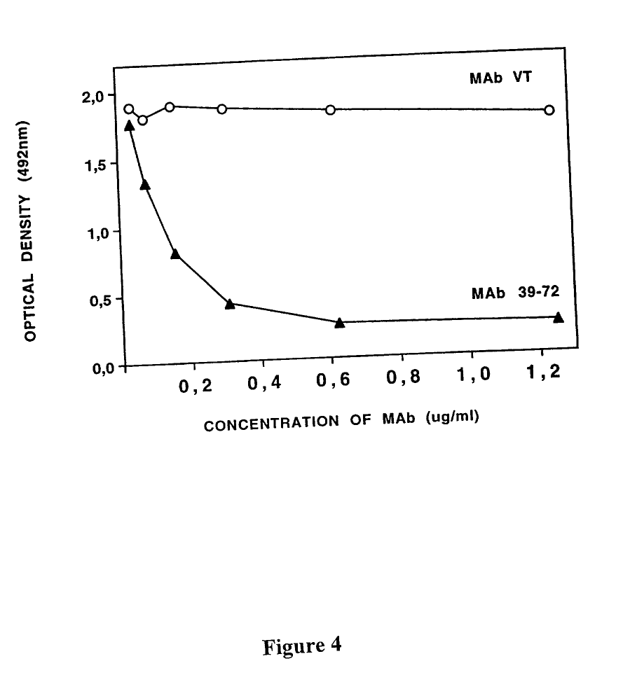

Naturally occurring core particles isolated from serum were used to induce anti-core MAbs. These new MAbs recognised epitopes mapping to aa sequence (3-68) of the core protein. One of these MAbs (MAb 1/1), being of IgM class, reacted with the recombinant NC 360 core protein and recognised an epitope located between amino acids (45-68), as determined using a panel of synthetic core peptides (FIG. 4A and B). This MAb was used for further studies.

Circulating HCV core particles contain HCV-RNA

Further experiments were carried out to confirm that: the core antigen-expressing particles were also present in native and unfractionated sera from HCV carriers, and that the core particles circulating in serum contained HCV-RNA. Fresh serum samples, that had never been frozen (to exclude the possibility of virion degradation) were analysed a few hours after blood sampling by affinity-capture RT-PCR. This method is based on the adsorption of viral particles by antibodies attached to PCR tubes. Viral RNA is then extracted, reverse transcribed and the cDNA amplified by PCR. The MAbs used to adsorb HCV core antigen-bearing particles were of the IgG (ACAP 27 ) and IgM (MAb 1/1) class to prevent possible false positive reactions due to the presence of rheumatoid factor in the serum of most of the HCV carriers. These experiments (FIG. 5) demonstrated that the antigenic sites that reacted with anti-core antibodies were already detectable in freshly collected, unfractionated serum samples, and were therefore not artefactually exposed by the fractionation procedure. They also demonstrate that at least a part of the HCV core antigen occurring naturally in serum was expressed on virus particles carrying HCV-RNA.

As the HCV core antigen was detected by direct ELISA in several unfractionated serum samples from HCV carriers, we subjected three such samples to affinity chromatography on Affigel columns with the anti-core MAbs ACAP27 bound to the solid support. HCV core antigen was eluted from the column, together with HCV-RNA. Virus particles 38-43 nm in diameter and larger particles of 54-62 nm in diameter, similar to those isolated from CsCl gradients, were observed in these preparations by electron microscopy (FIG. 3A).

Detection of HCV core antigen in serum in the presence of circulating anti-HCV antibodies

HCV core antigen was detected in several native and fractionated plasma and serum samples, despite the presence of circulating anti-HCV antibodies. We demonstrated, in competitive inhibition assays, that the reaction of mouse anti-core MAbs with the recombinant core protein was not inhibited by high concentrations (up to 200 μg/ml) of immunoglobulins isolated from the sera of HCV carriers (HCIG) (FIG. 6A). This preparation contained high levels of anti-core antibodies, as shown by routine assays and reactivity with synthetic core peptides (FIG. 1C). In contrast, homologous, unlabeled MAb ACAP-27, used as a control, inhibited this system at nanogram concentrations (FIG. 6B).

Further comparative analysis of the reactivity of anti-core antibodies of human and mouse origin was carried out by SPR (Bia-core) (FIG. 6C). Recombinant core protein was immobilised on the sensor chip and human anti-HCV globulins were then injected, followed by mouse MAbs. The binding of human antibodies (up 200 μg/ml) yielded only about 80 RU whereas the ACAP and VT MAbs, injected sequentially (at a concentration of 20 μg/ml) each showed strong binding (150 and 300 RU, respectively), despite the prior injection of HCIG (FIG. 6B). Complementary experiments performed with various concentrations of MAbs demonstrated an extremely high affinity for both MAbs (ACAP27 and VT) with an apparent dissociation constant Ka^{app} (5×10⁻¹¹ M and 1.6×10⁻¹³ M, respectively), much higher than that for human anti-core antibodies (2×10⁻⁷ M). Overall, these data suggest that HCV core antigen could be detected in the serum of HCV-infected patients, due to the large difference in affinity between the mouse MAbs, used in the detection assays, and circulating human anti-core antibodies.

Nucleocapsid-like particles are produced in insect cells infected with recombinant baculovirus

We investigated whether core particles similar to these isolated from human plasma were produced in insect cells infected in vitro with recombinant baculovirus. The HCV core protein was expressed in the infected Sf9 cells, as shown by western blotting (FIG. 7A), but no protein bands corresponding to HCV envelope proteins were detected in these cell extracts with anti-E1 (A2) and E2 (A11) MAbs (data not shown). No secretion of HCV proteins to the cell supernatants could be evidence by ELISA or Western blot. The soluble fraction was thereof ore obtained after the lysis of infected cells as previously described (3,4) and was subjected to isopyenic centrifugation in CsCl gradient. A major peak of core antigen was detected by ELISA at a density of 1.35-1.36 g/ml. (FIG. 7B). Nucleocapsid-like particles heterogeneous in size, ranging from 33 to 62 nm in diameter were observed in these fractions by electron microscopy, and were bound to microscope grids coated with anti-core antibody (FIG. 3 G). In a smaller peak of core antigen, at a density of 1.25 g/ml, virus particles 42-43 nm in diameter associated to fragments of membranes were observed by electron microscopy, and were also bound to anti-core MAbs coated grids (II).

Localisation of HCV core antigen in the liver of experimentally infected chimpanzees, using MAb 1/1

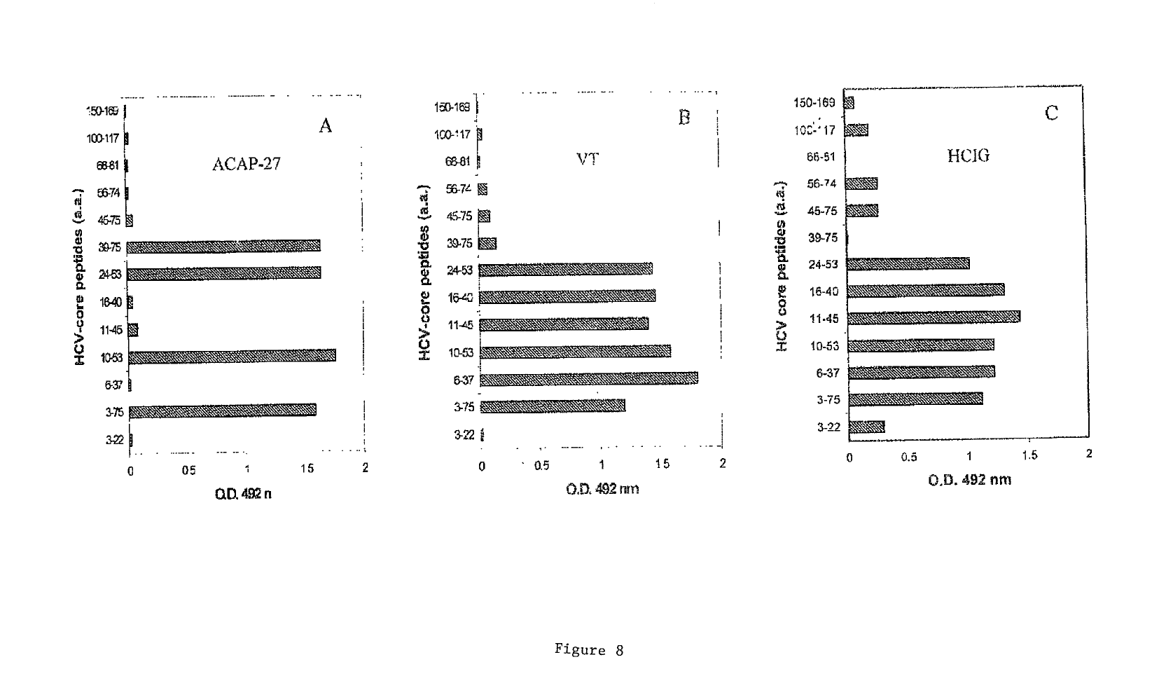

MAb 1/1, produced by immunisation with serum core particles, reacted with the core antigen in the liver of chimpanzees experimentally infected with HCV. Immunostaining of liver tissue obtained during the early and viraemic phase of the disease with MAb 1/1 resulted in granular fluorescence in the cytoplasm of hepatocytes. In CH1572, approximately 70% of liver cells contained small fluorescent granules, and 20% of hepatocytes showed much stronger granular and homogeneous fluorescence (FIG. 8A); in CH1537, the percentage of hepatocytes stained was similar but the fluorescence was less intense. The selective cytoplasmic nature of the fluorescence was confirmed by observations with a confocal laser microscope (FIG. 8 C). In liver specimens before inoculation, only a small number of powder-like granules of low-intensity fluorescence were identified in liver sinuses, sometimes in the close vicinity hepatocytes (FIG. 8 B). All other control specimens and immunochemical stainings were negative.

Detection of circulating HCV core antigen in serum of HCV infected chimpanzees.

Selected serum samples from CH1537 and CH1572 with HCV core in hepatocytes identified by immunohistochemistry were tested for the presence of circulating HCV core antigen by ELISA in the early phase of the infection. HCV core could be detected directly in serum of both chimpanzees: 38 days after inoculation in CH1537 and 28 days after inoculation in CH1572. ELISA OD readings were 4 to 5 standard deviation above the mean values of serum samples from the same chimpanzees before inoculation (negative controls). Both chimpanzees tested negative for circulating IICV core on day 52 and 45 respectively. Serum samples positive for core antigen contained IICV-RNA, but were negative for anti-core antibodies and were obtained substantially before a major peak of ALT: 39 days for chimpanzee 1537 and 46 days for chimpanzee 1572.

### Discussion

In this study, we show that virus particles that express on their surface core antigen occur naturally in the serum of HCV-infected individuals. These virus particles display physiochemical properties, antigenic reactivity and morphology similar to those of HCV nucleocapsids isolated by the treatment of putative HCV virions with detergent. The buoyant density of these virus particles (1.32-1.34 g/ml in CsCl) is that expected for non-enveloped, RNA-containing nucleocapsids. Indeed, using affinity RT-PCR, we confirmed that the HCV core antigen in serum was associated with HCV-RNA, and was therefore located on HCV RNA-bearing particles. Naturally occurring core particles were heterogeneous in size, with the predominant population 38-43 nm in diameter. Larger particles, 54-62 nm in diameter, were also consistently observed in core antigen preparations by electron microscopy and were also bound to the microscope grids by anti-core antibodies. Similar virus particles, mostly 37 to 43 nm in diameter, but also some larger, 54-62 nm particles, were observed by electron microscopy in preparations of viral nucleocapsids isolated by detergent treatment of putative IICV virions.

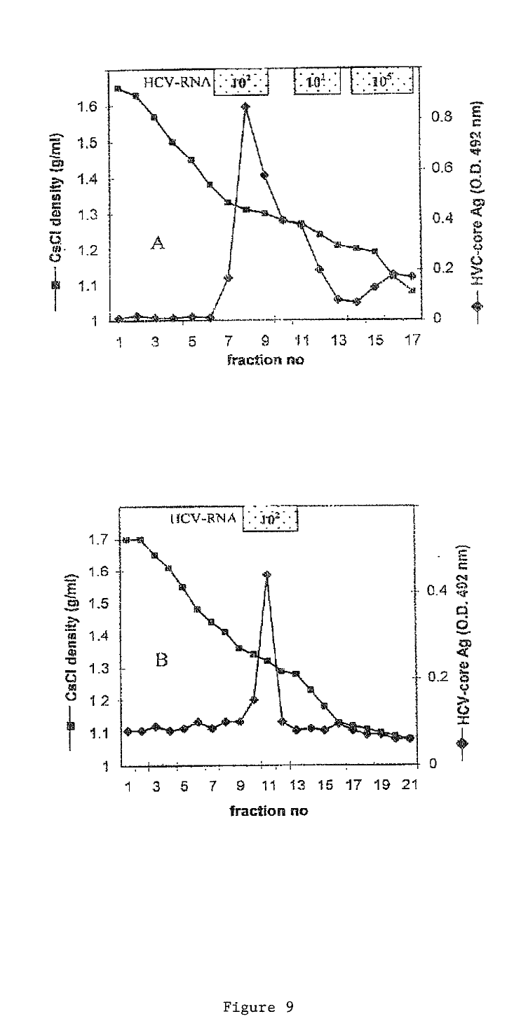

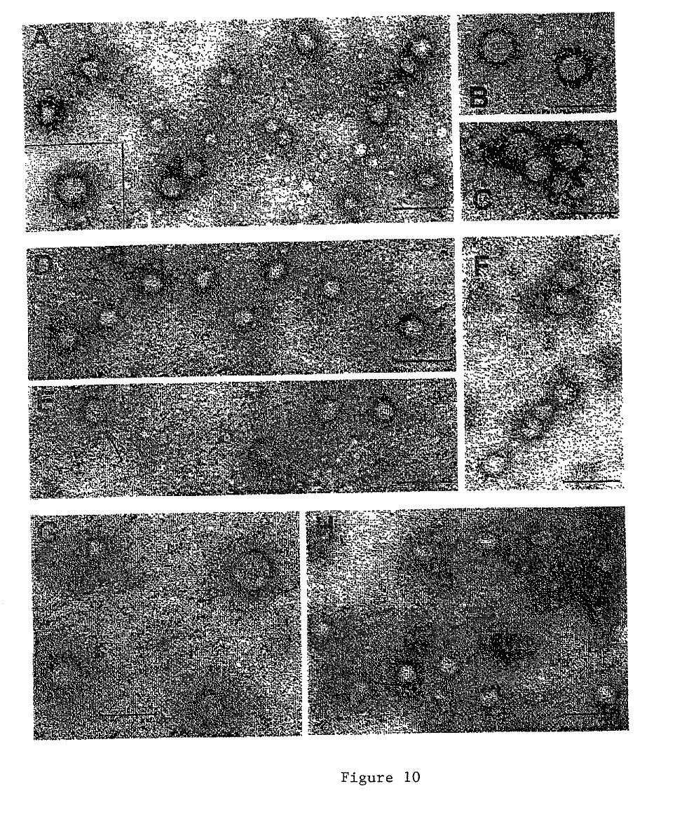

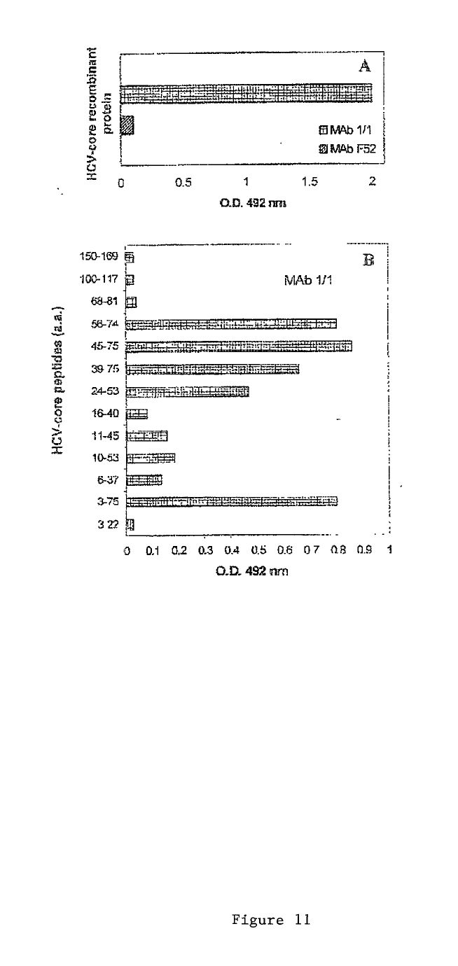

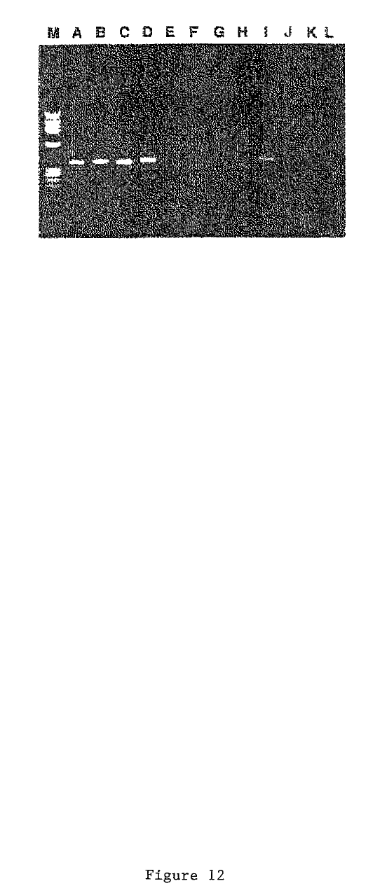

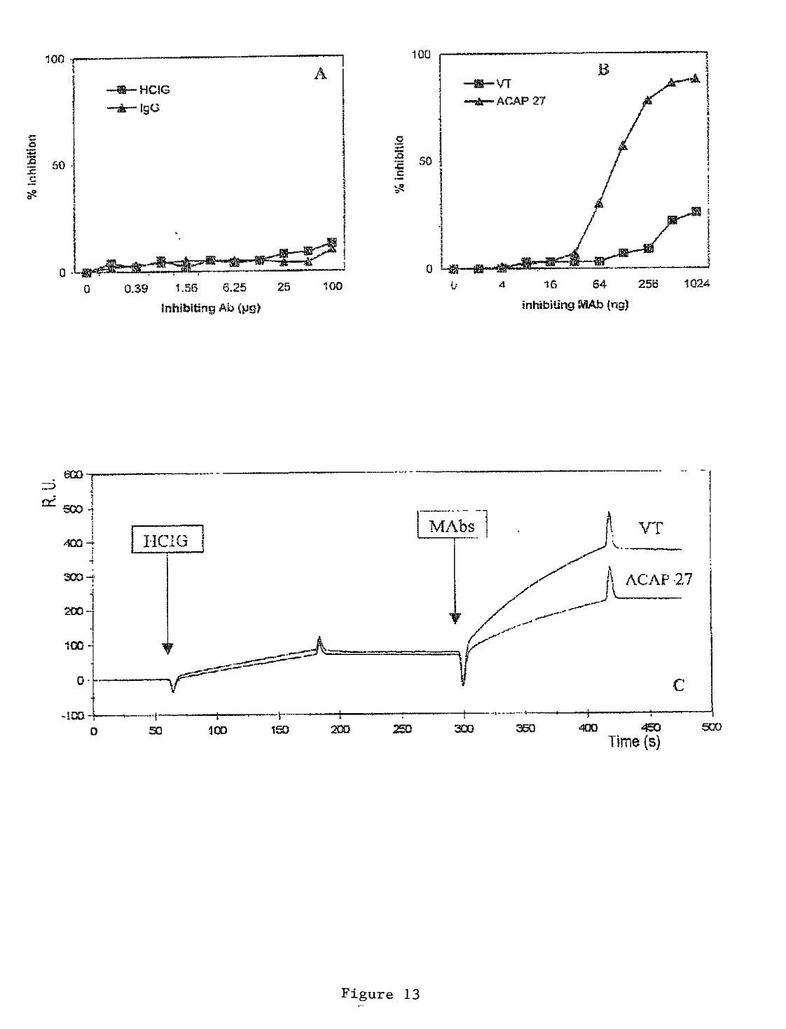

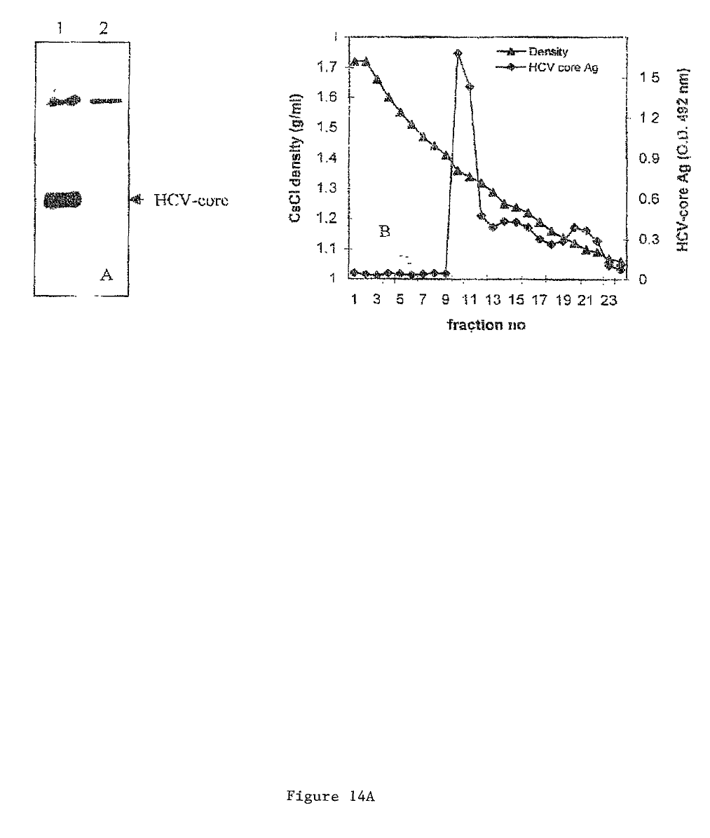

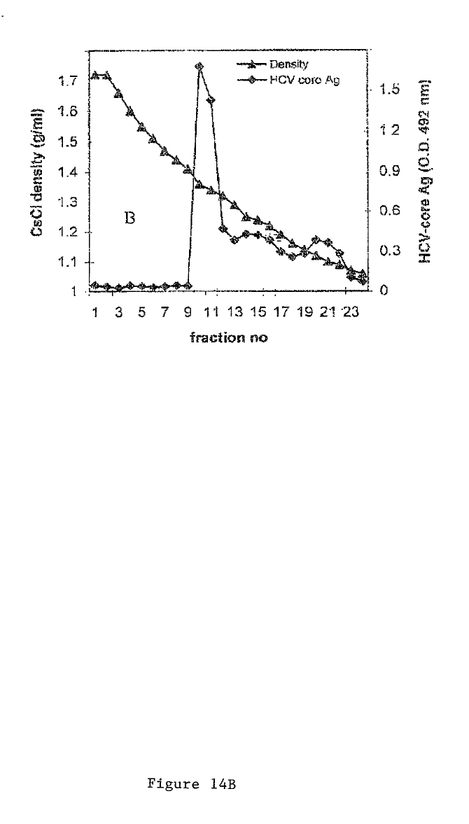

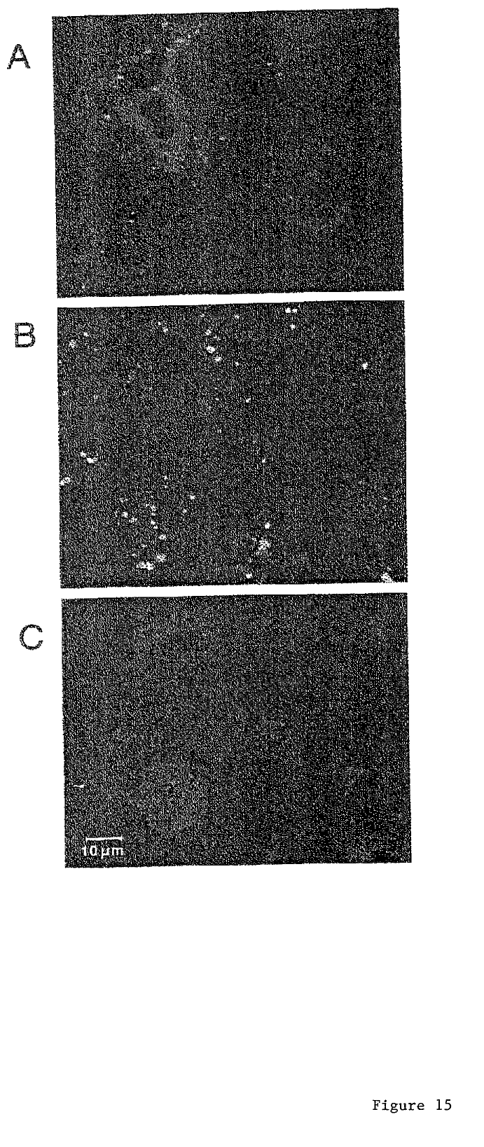

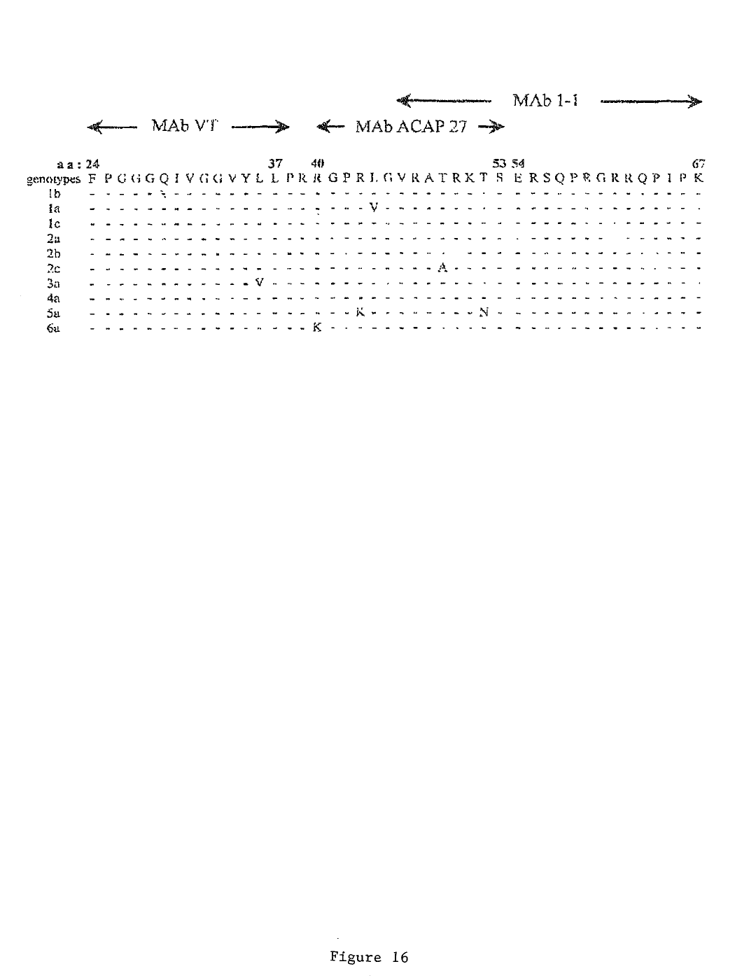

The principles of assays used in this study for detection of HCV core are different from all other assays published before (1,19,32,33,38,39,40) or recently commercialised (Ortho Diagnostics) which use detergents or denaturing agents for pre-treatment of serum samples or in a “sample diluent”. These assays quantify mainly the core protein from denatured HCV virions. In our study, HCV core antigen was detected directly by ELISA and by affinity RT-PCI in several, fresh, unfractionated and untreated serum and plasma samples. We were also able to isolate core particles directly from plasma by affinity chromatography with anti-core antibodies. Thus, core epitopes were not artefactually exposed by the fractionation procedure, but were instead naturally present on circulating HCV particles. At least three epitopes were found to map to the sequence between aa (24-68) of the serum core particle and this sequence seems to be well conserved in different HCV genotypes (FIG. 9).

Circulating core particles reacted with MAbs despite the presence of human anti-HCV antibody in the samples analysed. Indeed, human anti-core antibodies from the sera of HCV-infected individuals did not compete with mouse MAbs for these antigenic sites, probably due to the large differences in affinity demonstrated by SPR analysis. Therefore, HCV core antigen can be detected directly with immunological assays involving high-affinity MAbs, not only in the initial infection phase (window period) (33), but also, as shown here, during chronic disease, even if anti-HCV antibodies are present in the serum. The detection of circulating, envelope-free HCV nucleocapsids in serum has potential diagnostic applications (a patent is pending).

As the overproduction and release of nucleocapsids may be a feature of HCV morphogenesis, we investigated whether nucleocapsid-like HCV particles were also produced in insect cells infected with recombinant baculovirus containing c-DNA encoding the HCV core and envelope proteins. Indeed, a population of sub-viral particles was isolated from baculovirus-infected insect cells, that was reactive with anti-core MAbs. In accordance with previous observations (3,4) these particles were not secreted into cell culture supernatant, and a mild detergent treatment (the same as previously used by these authors to isolate enveloped virus-like particles) was required to isolate core-like particles from infected cells. These nucleocapsid-like particles banded at a density of 1.35-1.36 g/ml in CsCl gradient and were very heterogeneous in size (30-68 nm). The density of these particles suggested that they contained RNA, consistent with observations that the formation of nucleocapsid-like particles in vitro requires interaction of the core protein with RNA for encapsidation (22). Another population of core particles, isolated in this study from baculovirus-infected insect cells, banded at a density of 1.25 g/ml, was more homogeneous in size (42-43 nm) and co-sedimented with membrane fragments. These two populations of nucleocapsid-like particles may correspond to the two subpopulations of nucleocapsids reported for duck hepatitis β virus: cytosolic core particles, secreted from cells in a non-enveloped form and membrane-bound core particles, secreted from infected cells as enveloped virions (22). Although sub-viral, nucleocapsid-like particles has not yet been isolated from baculovirus-infected insect cells, Baumert et al. (4) reported that some of the virus-like particles produced in insect cells reacted with anti-core antidotes and stimulated anti-core antibody responses.

In this study, we generated new MAbs by immunisation of mice with HCV core particles naturally occurring in serum. One of these MAbs, used for the immunostaining of liver tissue from experimentally infected chimpanzees enabled to demonstrated the presence of HCV core antigen in the cytoplasm of hepatocytes at the acute and viraemic phase of the disease. In previous studies, polyclonal sera from IICV infected patients containing antibodies against several structural and non-structural recombinant HCV proteins have been used for immunostaining of HCV antigens in liver (21), but reactivity of these probes with HCV core in chimpanzee liver could not be evidenced by absorption studies. Moreover, the localisation of the core antigen in liver tissue at the acute phase of infection has never been demonstrated using MAbs. Most of these MAbs, induced by immunisation with synthetic or recombinant proteins (9,17,29) did not recognize liver HCV or reacted only with massive deposits of HCV antigens, in the livers of chronically infected chimpanzees (9,10,27). The reactivity of MAb 1/1 with the cytoplasm of hepatocytes indicates that either core protein, or core particles were accumulated in the liver cell at the early phase of infection.

Some previous observations have suggested that HCV core antigen-expressing viral structures may be present in the sera of HCV-infected individuals: a proportion of a high-density HCV population detected by RT-PCR was precipitated by anti-core antibodies (7,13,18), HCV core antigen has been detected in some serum samples by ELISA (25), and a few 45 nm nucleocapsid-like particles were observed by electron microscopy in the serum of an agammaglobulinaemic patient (42). Our data show clearly that the IICV riucleocapsid, which is thought to be present in the bloodstream as an internal component of infectious virions, is present in the sera of patients also as a free, non-enveloped particle, and is synthesised in large amounts in the baculovirus expression system in vitro. Therefore, the overproduction of HCV nucleocapsids and their release into serum seems to be a feature of HCV morphogenesis. The detection of core protein in immune complexes in the glomeruli of the kidneys of HCV-infected patients with membranous glomerulonephritis, in the absence of detectable E1, E2 and NS2/NS3 proteins in these deposits (31), is highly consistent with this notion and suggests that it is of physiological relevance in vivo.

Self-assembly of the HCV core protein, produced in bacteria, into nucleocapsid-like particles has been observed in vitro and it has been shown that this process requires interaction between the core protein and nucleic acid (22). This raises questions as to whether the circulating nucluocapsids described in this study contain complete HCV genome or some of them correspond to defective particles and whether some of these particles might be infectious.

Another question relates to whether HCV nucleocapsids are secreted from the infected cells in vivo or are released into bloodstream by damage of infected hepatocytes. HCV core particles characterised in this study were isolated mostly from plasma from volunteer blood donors with normal ALT levels and without any symptoms of liver injury. Although minor inflammatory changes can not be excluded in these patients, the presence of core particles in their serum did not correlate with liver damage. Moreover, analysis of serum samples from chimpanzees during the acute phase in infection, when liver biopsy specimens were taken (and before important elevation of transaminase levels) revealed the presence of circulating core antigen detectable by direct ELISA Although this question requires further studies, the observations reported herein suggest that non-enveloped nucleocapsids might be secreted from infected cells. The secretion of nucleocapsids devoid of envelope proteins has been reported for rhabdoviruses, retroviruses and, recently, for duck hepatitis B virus (24). HCV core protein was reported to be secreted from transfected hepatoma cell lines in culture and was detected in the serum of mice transgenic for the HCV core (23,35).

HCV is remarkably efficient at establishing and maintaining chronic infection and evolving mechanism to evade the host response. In addition to generating viral variants able to escape recognition by the humoral and cellular responses, it has been suggested that the HCV core protein plays a critical role in establishing HCV infection, by suppressing the immune response, particularly the production of virus-specific cytotoxic lymphocytes (CTL) and interferon in the early phase of infection (20,23). The overproduction and release of non-enveloped HCV nucleocapsids into the bloodstream and accumulation of the core protein(or core particles) in liver cells during an early phase of infection may be unconventional means by which HCV circumvents the host immune response and ensures its survival in the infected host.

Finally, attached to and incorporated into this disclosure are copies of the following publications:

1) Journal of General Virology, 74, 661-668 (1993), Simmond, et al;

2) “Detection et Caracterisation de la Nucleocapside du Virus de L'Hepatite C (VHC) Dans le Serum des Patients Infectes”, Mailard, P, et al;

3) “Analyse de la Structure Antigenique de Virus De L'Hepatite C (VCH)”, Budkowska et al;

4) Archives of Virology, “Ultrastructural and physiochemical characterization of the hepatitis C virus recovered from the serum of an agammaglobulinemic patient,” 143:2241-2245 (1993), Trestard et al;

5) Journal of Medical Virology, “Detection of Hepatitis C Virus Core protein Circulating Within Different Virus particle Populations, ” 55:1-6 (1998), Masalova et al;

Listed below of additional are citations for additional background publications.

REFERENCES (Background):

Hihahata M., Shimizu Y.K., Kato H., et al., “Equilibrium Centrifugation Studies of Hepatitis C Virus: Evidence for circulating Immune Complexes”, J. Virol., 67, 1953-1958, 1993;

Koshy, R.L., Inchauspe, G., “Evaluation of Hepatitis C Virus protein Epitopes for Vaccine Development, Trends in Biotechnology, 14, 364-369, 1996;

Thomssen, R., Bonk, S., Propfe C., Heerman, K.H., Köchel H.G., Uy, A., “Association of Hepatitis C Virus in Human Sera with β-lipoprotein”, Med. Microbiol. Immunol, 181, 293-300, 1992;

Takahashi K., Okamoto H., Kishimoto S., Munekata E., Tachibana K., Akahane Y., Yoshizawa H., and Mishiro S., “Demonstration of a Hepatitis C Virus Specific Antigen Predicted from the Putative Core Gene in the Circulation of Infected Host, ” J. Gen. Virol., 73, 667-672, 1992;

Takahaski, K., Kishimoto, s., Yoshizawa, H., Okamoto, H., Yoshikawa, A., and Mishiro, S., “p26protein and 33nm Particle associated with nucleocapsid of hepatitis C Virus Recovered from the Circulation of Infected Host, ” Virology, 191, 431-434, 1992.

### REFERENCES

1. Aoyagi, K., C. Ohue, K. Iida, T. Kimura, E. Tanaka, K. Kiyosawa, and S. Yagi. 1999Development of a simple and highly sensitive enzyme immunoassay for hepatitis C virus core antigen. journal of Clinical Microbiology 37:1802-1808.

2. Barba, G., F. Harper, T. Harada, M. Kohara, S. Goulinet, Y. Matsuura, G. Eder, Z. Schaff, M.J. Chapman, T. Miyamura, and C. Brechot. 1997. Hepatitis C virus core protein shows a cytoplasmic localization and associates to cellular lipid storage droplets. Proceedings of the National Academy of Sciences of the United States of America 94:1200-1205.

3. Baumert, T. F., S. Ito, D. T. Wong, and T. J. Liang. 1998. Hepatitis C virus structural proteins assembic into viruslike particles in insect cells. Journal of Virology 72:3827-3836.

4. Baumert, T. F., J. Vergalla, J. Satoi, M. Thomson, M. Lechmann, D. Herion, H. B. Greenberg, S. Ito, and T. J. Liang. 1999. Hepatitis C virus-like particles synthesized in insect cells as a potential vaccine candidate. Gastroenterology 117:1397-1407.

5. Bradley, D., K. McCaustland, K. Krawczynski, J. Spelbring, C. Humphrey, and E. H. Cook. 1991. Hepatitis C virus: buoyant density of the factor VIII-derived isolate in sucrose. Journal of Medical Virology 34:206-208.

6. Bradley, D. W., K. Krawczynski, J. W. Ebert, K. A. McCaustland, Q. L. Choo, M. A. Houghton, and G. Kuo. 1990. Parenterally transmitted non-A, non-B hepatitis: virus-specific antibody response patterns in hepatitis C virus-infected chimpanzees. Gastroenterology 99:1054-1060.

7. Chou, S. H., H. S. So, J. M. Cho, and W. S. Ryu. 1995. Association of hepatitis C virus particles with immunoglobulin: a mechanism for persistant infection. Journal of General Virology 76:2337-2341.

8. Deleersnyder, V.,A. Pillez, C. Wychowski, K. Blight, J. Xu, Y.S. Hahn, C.M. Rice, and J. Bibuisson. 1997. Formation of native hepatitis C virus glycoprotein complexes. Journal of Virology 71: 679-704.

9. Ferns, R. B., P. W. Tuke, and C. H. Sweenie. 1996. Characterisation of a panel of monoclonal antibodies raised against recombinant HCV core protein. Journal of Medical Virology 50: 221-229.

10. Gonzolez-Peralta, R. P., J. W. Fang, G. L. Davis, R. Gish, K. Tsukiyama-Kohara, M. Kohara, M. U. Mondelli, R. Lesniewski, M. Y. Phillips, M. Mizokami, and et al. 1994. Optimization for the detection of hepatitis C virus antigens in the liver [published erratum appears in j Hepatol jun. 20, 1994 (6):848]. Journal of Hepatology 20:143-147.

11. Grakoui, A., C. Wychowski, C. Lin, S. M. Feinstone, and C. M. Rice. 1993. Expression and identification of hepatitis C virus polyprotein cleavage products. Journal of Virology 67:1385-1395.

12. Han, J. Q., W. N. Schmidt, P. Wu, P. M. Loh, G. Neil, D. R. Labreckque, and J. T. Stapleton. 1997. Specific binding of hepatitis C virus to the Fc fragment of immunoglobulin molecules. Viril Hepatitis and Liver Disease, Rome:228-231.

13. Hijikata, M., Y. K. Shimizu, H. Kato, A. Iwamoto, J. W. shih, H. J. Alter, R. H. Purcell, and H. Yoshikura. 1993. Equilibrium centrifugation studies of hepatitis C virus: evidence for circulating immune complexes. Journal of Virology 67:1953-1958.

14. Hino, K., Fujii, M. Korenaga, C. Murakami, M. Okazaki, M. Okuda, and K. Okita. 1997. Correlation between relative number of circulating low-density hepatitis C virus particles and disease activity in patients with chronic hepatitis C. Digestive Diseasea & Sciences 42:2476-2481. p

15. Houghton, M. 1996. Hepatitis C virus, In B. N. Fields, D. M. Knipe, P. M. Howley et al. (ed.), Fields Virology, 3rd ed. Lippinot-Raven Publishers, Philadelphia. 1996:1035-1058.

16. Jin, D. Y., H. L. Wang, Y. Zhou, A. C. Chun, K. Y. Kibler, Y. D. Hou, H. Kung, and K. T. Jeang. 2000. Hepatitis C. virus core protein-induced loss of LZIP function correlates with cellular transformation. EMBO Journal 18:729-740.

17. Jolivet- Reynaud, C., P. Dalbon, F. Viola, S. Yvon, G. Paranhos-Baccala, N. Piga, I. Bridon, M. A. Trabaud, N. Battail, G. Sibai, M. Jolivet. 1998. HCV core immunodominant region anaylysis using mouse monoclonal antibidies and human sera: characterization of major epitopes useful for antigen detection. Journal Of Medical Virology 56:300-309.

18. Kanto, T., Hayashi, T. Takehara, H. Hagiwara, F. Mita, M. Naito, A. Kasahara, H. Fusamoto, and T. Kamada. 1995. Density analysis of hepatitis C virus particle population in the circulation of ifected hosts: implications for virus neutralization or persistannce. Journal of Hepatology 22:440-448.

19. Kashiwakuma, T., A. Hasegawa, T. Kijita, A. Tahata, H. Mori, Y. Ohta, E. Tanaka, K. KIyosawa, T. Tanaka, S. Tanaka, N. Hattori, and M. Kohara. 1996. Detection of hepatitis C virus specific core protein in serum of patients by a sensitive fluorescence enzyme immunoassay (FEIA). Journal of Immunological Methods 190:79-89.

20. KIttlesen, D. J., K. A. Chaianese-Bullock, Z. Q. Yao, T. J. Braciale, and Y. S. IIahn. 2000. Interaction between complement receptor gClqR and hepatitis C virus core protein inhibits T-lymphocyte proliferation. Journal of Clinical Investigation 106:1239-1249.

21. Krawezynski, K., M. J. Beach, D. W. Bradley, G. Kuo, A. M. di Bisceglie, M. Houghton, G. R. Reyes, J. P. Kim, Q. L. Choo, and M. J. Alter. 1992. Hepatitis C virus antigen in hepatocytes: immunomorphologic detection and identification. Gastroenterology 103:622-629.

22. Kunkei, M., M. Lorinezi, R. Rijnbrand, S. M. Lemon, and S. J. Watowich. 2001. Self-Assembly of Nucleocapsid-Like Particles from Recombinant Hepatitis C Virus Core Protein. J. Virol. 75:2119-2129.

23. Large, M. K., D. J. Kittlesen, and Y. S. Hahn. 1999. Soppression of host immune response by the core protein of hepatitis C virus: possible implications for hepatitis C virus persistence, Journal of Immunology 162:931-938.

24. Mabit, H., and H. Schaller. 2000. Intraccllular hepadnavirus nucleocapids are selected for secretion by envelope protein-independent membrane binding. Journal of Virology 74:11472-11478.

25. Masalova, O. V., S. N. Atanadze, E. I. Samokhvalov, N. V. Petrakova, T. I. Kalinina, V. D. Smirnoc, Y. E. Khudyakov, H. A. Fields. and A. A. Kushch. 1998. Detection of hepatitis C virus core protein circulating within different virus particle populations. Journal of Medical Virology 55:1-6.

26. Matsumoto, M., T. Y. Hsieh, N. Zhu, T. VanArsdale, S. B. Hwang, K. S. Jeng, A. E. Gorbalenya, S. Y. Lo, J. H. Ou, C. F. Ware, and M. M. Lai. 1997. Hepatitis C virus core protein interacts with the cytoplasm tail of lymphotoxin-beta receptor. Journal of Virology 71:1301-1309.

27. McLauchlan, J. 2000. Properties of the hepatitis C virus core protein: a structural protein that modulates cellular processes. Journal of Viral Hepatitis 7:2-14.

28. Miyamoto, H., H. Okamoto, K. Sato, T. Tanaka, and S. Mishiro. 1992. Extraordinarily low density of hepatitis C virus estimated by source density gradient centrifugation and the polymerase chain reaction. Journal of General Virology 73:715-718.

29. Moradpour, D., T. Wakita, K. Tokushige, R. I. Carlson, K. Krawczynski, and J.R. Wands. 1996. Charaterization of three novel monoclonal antibodies against hepatitis C virus core protein. Journal of Medical Virology 48:234-241.

30. Nolandt, O., V. Kern, H. Muller, E. Pfaff, L. Theilmann, R. Welker, and H. G. Krausslich. 1997. Analysis of hepatitis C Virus core protein interaction domains. Journal of General Virology 78:13331-1340.

31. Okada, K., Y. Takishita, H. Shimomura, T. Tsuji, T. Miyamura, T. Kuhara, K. Yasutomo, S. Kagami, and Y. Kuroda. 1996. Detection of hepatitis C virus core protein in the glomeruli of patients with membranous glomerulonephirtis. Clinical Nephrology 45:71-76.

32. Orito, E., M. Mizokami, T. Tanaka, J. Y. Lau, K. Suzuki, M. Yamauchi, Y. Ohta, A. Hasegawa, S. Tanaka, and M. Kohara. 1996. Quantification of serum hepatitis C virus core protein level in patients chronically infected with different hepatitis C virus genotypes. Gut 39:876-880.

33. Peterson, J., G. Green, K. lida, B. Caldwell, P. Kerrison, S. Bernich, K. Aoyagi, and S. R. Lee. 2000. Detection of hepatitis C core antigen in the antibody negative ‘window’ phase of hepatitis C infection. Vox Sanguinis 78:80-85.

34. Prince, A. M., T. Huima-Byron, T. S. Parker, and D. M. Levine. 1996. Visualization of hepatitis C virions and pulative defective interfering particles isolated from low-density lipoproteins. Journal of Viral Hepatitis 3:11-17.

35. Sabile, A., G. Perlemuter, F. Bono, K. Kohara, F. Demaugre, M. Kohara, Y. Matsupra, T. Miyamura, C. Brechot, G. Barba. 1999. Hepatitis C virus core protein binds to apolipoprotein AII and its Secretion is Modulated by fibrates. Hepotology 30:1064-1076.

36. Santolini, E., GMigliaccio, and N. la Monica. 1994. Biosynthesis and biochemical properties of the hepatitis virus core protein. Journal of Virology 68:3631-3641.

37. Shivastova, A., S. K. Manna, R. Ray, and B. B. Aggarwal. 1998. Ectopic expression of hepatitis virus core protein differentially regulates nuclear transcription factors. Journal of Virology 72:9722-9728.

38. Takahashi, K., S. Kishimoto, H. Yoshizawa, H. Okamoto, A. Yoshikawa, and S. Mishiro. 1992. p.26 protein and pb 33-nm particle associated with nucleocapsid of hepatitis C virus recovered form the circulation of infected hosts. Virology 191:431-434.

39. Takahashi, K., H. Okamoto, S. Kishimoto, E. Munekata, K. Tachibana. Y. Akahane, H. Yoshizawa, and S. Mishiro. 1992. Demonstration of a hepatitis C virus-specific antigen predicted form the pulative core guide in the circulation of the infected hosts. Journal of General Virology 73:667-672.

′. Tanaka, E., K. Kiyosawa, A. Matsumoto, T. Kashiwakuma, A. Hasegawa, H. Mori, O. Yanagihara, and Y. Ohta. 1996. Serum levels of hepatitis C virus core protein in patients with chronic hepatitis C treated with interferon alfa. Hepatology 23:1330-1333.

41. Thomssen, R., Bonk, C. Propfe, K. H. Heermann, H. G. Kochel, and A. Uy. 1992. Association of hepatitis C virus in human sera with beta-lipoprotein. Medical Microbiology & Immunology 181:239-300.

42. Trestard, A., Y. Bacq, L. Biizelay, F. Dubois, F. Barin, A. Goudeau, and P. Roingeard. 1998. Ultrastructural and physicochiemical characterization of the hepatitis C virus recovered from the: serum of an agammaglobulinemic patient. Archives of Virology 143:2241-2245.

43. Yasui, K., T. Wakita, K. Tsuiyama-Kohara, S. I. Funhashi, M. Ichikawa, T. Kajita, D. Moradpour, J. R. Wands, and M. Kohara. 1998. The native form and maturation process of hepatitis C virus core protein. Journal of Virology 72:6048-6055.

44. Zhu, N., A, Khoshnan, R. Schneider, M. Matsumoto, G. Dennert, C. Ware, and M. M. Lai. 1998. Hepatitis C virus core protein binds to the cyloplasmic domain of tumor necrosis factor (TNF) receptor 1 and enhances TNF-induced apoptosis. Journal of Virology 72:3691-3697.

What is claimed is:

1. A set of primers selected from the group consisting of (1) and (2); and (3) and (4):
1: CAT(or C).GTA (or G).AGG.GTA.TCG.ATG.AC, and
2: ATC.GCC.TGA.TAG.GGT.GCT.TGC.GAG; and
3: AGG.TCT.CGT.AGA.CCG.TGC.ATC.ATG, and
4: TTG.CG.G(or T)G (or C).A.CCT.A(or T)CG.CCG.GGG.GTC.

2. A method of directly detecting Hepatitis C virus in a fractionated or non-fractionated serum of a patient by detecting said virus with primers corresponding to viral RNA encoding core protein, said RNA being a light fraction of the total viral RNA, said light fraction being isolated after ultracentrifugation in a CsCl gradient of human serum containing HCV virus, and containing most of the circulating infectious HCV virus particles, the process comprising precipitating RNA, then effecting reverse transcription, and then effecting amplification with the set of primers of claim 1.

3. A method of detecting non-enveloped nucleocapsid or non-enveloped core protein of HCV in serum of patients, which comprises:
a) contacting the native serum of patients, optionally treated by chemicals or by physical process or fractionation, with monoclonal antibodies recognizing core protein or nucleocapsid protein;
b) eliminating compounds of the reaction not forming an immune complex with the monoclonal antibodies and which undergo non-specific reaction; and
c) detecting the immune complex formed between the core protein or the nucleocapsid protein and a monoclonal antibody by adding a second monoclonal antibody recognizing the core protein, the second monoclonal antibody being labeled by a radioactive or a non-isotopic marker.

4. The method of claim 3, wherein said non-isotopic marker is a fluorescent or enzymatic marker.

5. The method of claim 3, wherein the serum of the patient is fractionated by:
a) precipitating serum by PEG (polyethyleneglycol);
b) centrifuging the pellet after precipitation containing the HCV virus; resuspending the same in a buffer;
c) ultra-centrifuging the viral suspension in a CsCl gradient (cesium chloride gradient) optionally containing proteases inhibitors; and
d) testing each fraction.

6. The method of claim 3, wherein in step b), said compounds not forming said immune complex or forming non specific complex comprise rheumatoid factor.

7. A diagnostic kit for HCV infection, comprising at least:
a) a solid phase on which antibodies against nucleocapsid or core protein are coated;
b) a sample of purified core protein or nucleocapsid protein of HCV;
c) a sample of monoclonal antibodies labeled and unlabeled specific of HCV protein and, if necessary, a substrate to reveal the marker;
d) a sample of negative human serum;
e) a sample of positive control serum; and optionally
f) buffers and chemicals for testing said serum.

8. A purified polyclonal or a purified monoclonal antibody, which reacts with epitopes of core protein of HCV and which are not bound by human antibodies in the serum of patients and which react with the nucleocapsid or the core protein in infected tissues.

9. A hybridoma deposited at the C.N.C.M. on Apr. 14, 1999, under accession number I-2183.

10. The method of claim 3, which comprises:
a) separating nucleocapsid or core protein from the remaining compounds present in the serum which interfere with the detection of the nucleocapsid in tissues or in serum; and
b) detecting the same with polyclonal or monoclonal antibodies recognizing the non-enveloped antigens of HCV.

11. A method of detecting the presence of HCV particles in patient's serum or plasma without chemical pretreatment comprising:
contacting a patient's serum to be tested with a solid phase coated with a first antibody which is directed against HCV core protein;
adding at least one second labeled antibody directed against the HCV core protein, wherein said second antibody can be the same or different from said first antibody; and
detecting the presence or absence of a immune complex formed between said first antibody, said HCV core protein, and said second antibody wherein the presence of said immune complex indicates the presences of HCV particles in the patient's serum or plasma.

12. The method according to claim 11, wherein said second antibody is a mixture of at least two antibodies directed against the HCV core particle.

13. The method according to claim 11, wherein the secondary antibody is selected from the group consisting of
an antibody recognizing the amino acid region 24-37 of the core protein;
an antibody recognizing the amino acid region 40-53 of the core protein; and
an antibody recognizing the amino acid region 45-68 of the core protein.

14. The method according to claim 11, wherein said second antibody is enzymatically labeled.

15. The method according to claim 11, wherein the second antibody is labeled with peroxidase.

16. The method according to claim 12, wherein the second antibody is labeled with β-galactosidase.

17. The method according to claim 11, wherein the second antibody is labeled with alkaline phosphatase.

18. The method according to claim 11, wherein the second antibody is labeled with a radioactive marker.

19. The method according to claim 11, wherein the second antibody is labeled with a fluorescent marker.

20. A method of preparing a nucleocapsid-like particle recognized by at least one antibody selected from the group consisting of
an antibody recognizing the amino acid region 24-37 of the core protein;
an antibody recognizing the amino acid region 40-53 of the core protein; and
an antibody recognizing the amino acid region 45-68 of the core protein, wherein said process comprises:
introducing a HCV-1 core gene into a eukaryotic host cell;
culturing the transfected eukaryotic cell for under conditions suitable for the expression of said HCV- 1 core gene; and
separating a nucleocapsid-like particle from said host cell.

21. The method according to claim 20, wherein said introducing comprises transfecting said eukaryotic cell with a plasmid carrying said HCV-1 core gene.

22. The method according to claim 20, wherein said introducing comprises infecting said eukaryotic cell with a baculovirus carrying said HCV-1 core gene.

23. A nucleocapsid-like particle produced by the method of claim 20.

24. A purified monoclonal or polyclonal antibody which binds to the nucleocapsid-like particle of claim 23.

25. A detection kit for HCV infection comprising:
at least one antibody according to claim 24; and
reagents for labeling and visualization of a reaction between the serum or plasma of a patient to be tested for HCV infection and said at least one antibody.

26. The detection kit of claim 25, wherein said detection kit further comprises a negative control reagent.

27. The detection kit of claim 25, wherein said detection kit further comprises a positive control reagent.
